(19) United States
(12) Patent Application Publication
(10) Pub. No.: US 2007/0148660 A1
(43) Pub. Date: Jun. 28, 2007

(54) Treatment of maladaptive substance use with H-ras antagonists

(75) Inventor: Dorit Ron, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/453,560

(22) Filed: Jun. 14, 2006

Related U.S. Application Data
(60) Provisional application No. 60/692,006, filed on Jun. 16, 2005.
(60) Provisional application No. 60/694,467, filed on Jun. 27, 2005.

(51) Int. Cl. C12Q 1/68; G06F 19/00

(52) U.S. Cl. 435/6; 702/20

(57) ABSTRACT

The present invention provides a method of mitigating a symptom of maladaptive substance use in a subject by inhibiting H-ras. In addition, the invention provides a variety of prescreening and screening methods aimed at identifying agents that modulate a symptom of maladaptive substance use. Methods of the invention can involve assaying test agent binding to H-ras. Alternatively, test agents can be screened for their ability to alter the level of H-ras polypeptides, polynucleotides, or function. Finally, the invention also provides a diagnostic method that entails measuring one or more of these levels and determining risk for maladaptive substance use based on comparison to the corresponding level for a control population.

### CROSS-REFERENCE TO RELATED APPLICATIONS

This application claims the benefit of U.S. Provisional Application No. 60/629,006, filed Jun. 16, 2005, and U.S. Provisional Application No. 60/694,467, filed Jun. 27, 2005, each of which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under National Institute on Alcohol and Drug Abuse and Alcoholism Grant RO1AA013438-03. The Government may have certain rights in the invention.

### FIELD OF THE INVENTION

This invention pertains to methods of mitigating a symptom of maladaptive substance use, such as symptoms associated with maladaptive use of opioids, psychostimulants, cannabinoids, empathogens, dissociative drugs, and ethanol, as well as to related compositions and screening and diagnostic methods.

### BACKGROUND OF THE INVENTION

N-methyl D-aspartate receptors (NMDARs) are major mediators of processes such as synaptic plasticity and learning and memory (1), and also of pathological states such as schizophrenia, seizures, pain and drug and alcohol addiction (2-5), and alcohol intoxication (6).

NMDARs are heteromeric ligand-gated ion channels composed of an obligatory NR1 subunit and regulatory NR2A-D and NR3 subunits (2). The NMDAR subunits can undergo lateral movement between synaptic and extrasynaptic sites, as well as internalization from, or forward trafficking to, the plasma membrane (7,8). For example, prolonged inhibition of NMDAR activity results in the redistribution of the subunits from extrasynaptic to synaptic sites (9). Synaptic activity and long-term potentiation (LTP) increase the forward trafficking of NMDAR subunits (10,11), and glycine has been reported to prime the NMDAR subunits for internalization (12).

NMDAR function can be regulated by changes in the phosphorylation-state of the subunits via the activation of kinases or phosphatases (13). Protein phosphorylation and dephosphorylation also control the movement of NMDAR subunits to and from the plasma membrane. For example, activation of protein kinase C (PKC) and cAMP-dependent protein kinase A (PKA), and the consequent phosphorylation of the NR 1 subunit, increases forward trafficking of the receptor (14). Fyn kinase-mediated tyrosine phosphorylation of the NMDAR plays a role in the dopamine D1 receptor-mediated redistribution of NMDAR subunits from intracellular to postsynaptic compartments (15). Conversely, tyrosine dephosphorylation of the NR2A subunits leads to the internalization of the subunits, resulting in the rundown of channel activity (16). In addition, H-ras-mediated inhibition of Src kinase activity prevents the phosphorylation and membrane retention of NR2A subunits (17).

The activity of the NMDAR in the presence of ethanol is modulated via changes in the phosphorylation-state of the subunits (18-20). NMDARs have been implicated in mediating ethanol-associated phenotypes such as tolerance, dependence, withdrawal, craving and relapse, and intoxication (5,6); however, the mechanisms by which ethanol's actions on the NMDAR underlie these phenotypes are not well understood.

### SUMMARY OF THE INVENTION

The invention provides a method of mitigating a symptom of maladaptive substance use, wherein the method entails administering an effective amount of an inhibitor of H-ras to a subject, whereby the symptom of maladaptive substance use is mitigated.

Another aspect of the invention is a pharmaceutical composition comprising an inhibitor of H-ras and a pharmaceutically acceptable carrier.

The invention also provides a method of prescreening for an agent that can modulate a symptom of maladaptive substance use in a subject. In one embodiment, the method entails: (a) contacting a test agent with a H-ras; (b) determining whether the test agent specifically binds to H-ras; and (c) if the test agent specifically binds to H-ras, selecting the test agent as a potential modulator of a symptom of maladaptive substance use in a subject. In another embodiment, the method entails: (a) contacting a test agent with a polynucleotide encoding H-ras; (b) determining whether the test agent specifically binds to the polynucleotide encoding H-ras; and (c) if the test agent specifically binds to the polynucleotide encoding H-ras, selecting the test agent as a potential modulator of a symptom of maladaptive substance use in a subject.

Also provided is a method of screening for an agent that can modulate a symptom of maladaptive substance use in a subject. This method entails (a) contacting a test agent with a H-ras; (b) determining whether the test agent acts as an agonist or an antagonist of H-ras; and (c) if the test agent acts as an agonist or antagonist of H-ras, selecting the test agent as a potential modulator of maladaptive substance use in a subject.

In another embodiment, the invention provides an in vivo method of screening for an agent that that can modulate maladaptive substance use in a subject. This method entails (a) selecting a modulator of a H-ras as a test agent; and (b) measuring the ability of the selected test agent to modulate a symptom of maladaptive substance use in an animal model.

Another aspect of the invention is a method of assessing a subject's risk for maladaptive substance use. This method entails determining the level of H-ras polypeptides, polynucleotides, or function in a biological sample from the subject, wherein risk for maladaptive substance use is directly correlated with that level.

The invention also provides kits. In one embodiment, a kit of the invention includes: (a) an inhibitor of a H-ras in a pharmaceutically acceptable carrier; and (b) instructions for carrying out a method of the invention for mitigating a symptom of maladaptive substance use. In another embodiment, a diagnostic kit of the invention includes: (a) a component that specifically binds to a H-ras polypeptide or polynucleotide; and (b) instructions for carrying out a method of the invention for assessing a subject's risk for maladaptive substance use.

### BRIEF DESCRIPTION OF THE DRAWINGS

FIG. 1. Ethanol alters membrane surface expression of NR2A subunits. (a) Hippocampal slices were treated with artificial cerebrospinal fluid (aCSF; Ctl) or with 100 mM ethanol for 15 min, at room temperature followed by treatment with aCSF ((−)=total, lanes 1,3) or cross-linker (BS³=internalized pool, lanes 2,4) as described in methods. Samples were analyzed by western blot with anti-NR2B (left), anti-NR2A (right) and anti-actin antibodies. *p<0.05, n=4, (one-way ANOVA). (b) L(−tk) cells expressing (left) NR1+NR2B or (right) NR1+NR2A were treated with aCSF (Ctl) or with 100 mM ethanol for 15 min, at room temperature followed by treatment with aCSF ((−)=total, lanes 1,3) or cross-linker (B S₃=internalized pool, lanes 2,4). Samples were analyzed by Western blot with anti-NR2B (left), anti-NR2A (right) and anti-actin antibodies. *p<0.05, n=6, (Student's t-test). (c-d) Hippocampal slices were treated with aCSF (Ctl) or with 100 mM ethanol for 15 min, at room temperature followed by treatment with aCSF ((−)=total, lanes 1,3) or cross-linker (BS³=internalized pool, lanes 2,4) as described in methods. Samples were analyzed by Western blot with (c) anti-NR1, (d) anti-GluR1, and anti-actin antibodies (c,d). n=4. (e) Hippocampal slices were treated with aCSF (Ctl) or with 25 or 50 mM ethanol for 15 min at room temperature followed by treatment with aCSF ((−)=total, lanes 1,3,5) or cross-linker (BS³=internalized pool, lanes 2,4,6). Samples were analyzed by Western blot with anti-NR2A and anti-actin antibodies. **p<0.01, n=3-5, (One-way Anova). (f) Hippocampal slices were treated with aCSF (Ctl) or with 100 mM ethanol for 15 min at room temperature. After ethanol treatment, slices were washed twice with aCSF, and then recovered for an additional 15 min, and treated with aCSF ((−)=total, lanes 1,3) or cross-linker (BS³=internalized pool, lanes 2,4). Samples were analyzed by Western blot with anti-NR2A and anti-actin antibodies. Ctl vs Eth, **p<0.01; Eth vs washout, #p<0.01, n=7, (One-way Anova). (g) Hippocampal slices treated with aCSF (Ctl) or with 100 mM ethanol (E) for 15 min at room temperature followed by treatment with aCSF ((−)=total, lanes 1,3) or chymotrypsin (Chy=internalized pool, lanes 2,4). Samples were analyzed by Western blot with anti-NR2A and anti-actin antibodies. **p<0.01, n=3, (Student's t-test). For all experiments, the band intensity of NR2 subunits was normalized to actin, and histogram shows ratios of the internalized (BS³ or Chy) to total NR2A or NR2B plotted as means±S.D. The n values are the number of individual experiments from individual animals. There were no changes in the total amount of protein in the samples, and equal amounts of proteins were loaded in each lane.

FIG. 2. Ethanol exposure increases the association of the NR2A subunits with adaptin β2. (a) Hippocampal slices were treated with aCSF (control, lane 1), 100 mM ethanol for 15 min at room temperature (lane 2), or 100 mM glycine for 5 min (lane 3). Slices were homogenized as described in methods, and NR2A and adaptin β2 were immunoprecipitated (IP-ed) with 5 μg of (left) anti-NR2A, or (right) anti-adaptin β2 antibodies (top panels, IP). Co-IPs of (left) adaptin β2 or (right) NR2A were determined by immunoblotting with anti-adaptin β2 or anti-NR2A antibodies (bottom panels, IB). Data are expressed as the ratio of NR2A-adaptin β2 association to the IP of either NR2A or adaptin β2, and calculated as the % increase in NR2A-adaptin β2 association over control and plotted as means±S.D. *p<0.05, n=4, (Student's t-test). (b) L(−tk) cells expressing (left) NR1+NR2A or (right) NR1+NR2B and PSD95 as described in methods, were treated with aCSF (control, lane 1) or 100 mM ethanol for 15 min at room temperature (lane 2). The cells were homogenized as described in methods, and NR2A, or NR2B were IP-ed with 5 μg of (left) anti-NR2A, or (right) NR2B antibodies (top panels, IP). Co-IP of adaptin β2 was determined by immunoblotting with anti-adaptin β2 antibodies (bottom panels, IB), n=3. (c) Hippocampal slices were treated with aCSF (lane 1), or 100 mM ethanol (lane 2) for 15 min at room temperature. Slices were homogenized and adaptin β2 was IP-ed with anti-adaptin β2 antibodies (top panel, lanes 1,2). Co-IPs of TrkB (middle panel), or GluR1 (bottom panel) were determined by immunoblotting with anti-TrkB and antiGluR1 antibodies n=3. Also shown are the input (lane 3), and a control for the immunoprecipitation experiments (lane 4). The n values for all the experiments are the number of individual experiments from individual animals. There were no changes in the total amount of protein in the samples.

FIG. 3. Ethanol increases Ras activity and dominant negative Tat-H-Ras inhibits ethanol-mediated NR2A endocytosis and internalization. (a) Hippocampal slices were treated with aCSF (control, lane 1), 100 mM ethanol for 15 min (lane 2), or 50 mM KCl for 5 min (lane 3), at room temperature. Ras activity was determined with Raf-1-Ras binding domain GST-agarose binding assay as described in methods. Data are expressed as the ratio of Ras-GTP over total Ras levels, normalized to the control. **p<0.01, (Student's t-test). (b) Hippocampal slices were pre-treated for 2 hrs with aCSF (lanes 1,2) or with 2 μM Tat-HRasDN (lane 3), and 2 μM of the Tat-peptide (lane 4) at room temperature. Slices were then treated with aCSF (lane 1, control), 100 mM ethanol for 15 min (lane 2-4), and Ras activity was determined as described above. n=2. (c) Hippocampal slices were treated with aCSF (control, lane 1), 100 mM ethanol for 15 min (lane 2), 2 μM Tat-H-Ras DN for 2 hrs (lane 3), or Tat-H-Ras DN followed by 100 mM ethanol for 15 min (lane 4), as described in (b). Immunoprecipitations were conducted as described in FIG. 2, and Co-IPs were determined by probing the membranes with anti-NR2A (left) and anti-adaptin β2 antibodies (right). Data are expressed as % increase over control in the ratio of co-IPed protein to IPed protein. For IP of NR2A (left), Ctl vs Eth, *p<0.05; Eth vs Tat-H-RasDN+Eth, #p<0.05, n=4, For IP of Adaptin (right), Ctl vs Eth, *p<0.05; Eth vs Tat-H-RasDN+Eth, #p<0.05, n=4, (Student's t-test). (d) Following treatment as described for (c), the slices were incubated with either aCSF (−) (lanes 1,3,5,7) or cross-linker (B S3) (lanes 2,4,6,8), the levels of NR2A subunit were determined and analysis was performed as described in FIG. 1. Data are expressed as means±S.D. Ctl vs Eth, *p<0.05; Eth vs Tat-H-RasDN+Eth, #p<0.05, n=3, (One-way ANOVA).

FIG. 4. Inhibition of Src kinase is necessary for ethanol-mediated NR2A internalization. (a) Hippocampal slices were treated with aCSF (control, lane 1) or with 100 mM ethanol for 15 min (lane 2). IP was conducted using 5 μg of anti-Src or control anti-IgG antibodies (lane 4). Also shown is the input (lane 3). The membranes were probed with anti-[pY4 18]Src (top) and anti-Src (bottom) antibodies. The bar histogram of shows ratios of anti-[pY4 18]Src to total Src, *p<0.05, n=3, (Student's t-test). (b-c) Hippocampal slices from (b) (left) Src+/+, (right) Src+/− and (c) Fyn−/− mice were treated with aCSF (Ctl) or with 100 mM ethanol (lanes 3, 4) for 15 min at room temperature, followed by cross-linking as described in FIG. 1. Membranes were probed with anti-NR2A and anti-actin antibodies. Analysis was performed as described in FIG. 1, and the data are normalized to actin, and surface expressed or internalized to total NR2A subunits, plotted as means±S.D. of at least 3 experiments. (b) Src+/+ *p<0.05, n=3, (Student's t-test). (c) Fyn−/− **p<0.01, n=5, (Student's t-test).

FIG. 5. Ethanol exposure alters functional NMDAR properties. (a) NMDAR-mediated fEPSPs measured in the absence (11 slices from 8 mice) or presence (8 slices from 4 mice) of 1 μM Tat-H-Ras DN (which was pre-incubated with slices for 2 hr), followed by the application of 80 mM ethanol as indicated by the horizontal line. Inset shows example traces of NMDAR fEPSPs in control (left) and Tat-H-Ras DN treated slices (right) before (a) and during (b) application of ethanol. (b) Bar histogram of NMDAR fEPSPs at 13 min (peak inhibition by ethanol) and 25 min (5 min after ethanol washout) as shown in (a). *p<0.05 (One-way ANOVA). (c) Decay of NMDAR EPSCs were measured before, during and upon washout of 80 mM of ethanol. *p<0.05 compared to time 0 paired t-test, n=6 cells from 5 mice. (d) Inset shows example traces of NMDAR-mediated EPSCs at each time point. (e) NMDAR-mediated EPSCs measured before and during application of 0.4 mM NVP-AAM077 (white bar) and ethanol (gray bar) in 1 min bins; n=6 cells from 5 mice. Inset shows example traces of NMDAR-mediated EPSCs before (control), in the presence of 0.4 μM NVP-AAM077, and 20 min after application of ethanol in the presence of NVP-AAM077. (f) NMDAR-mediated EPSCs measured before and during application of 3 μM ifenprodil (white bar) and ethanol (gray bar) in 1 min bins; n=7 cells from 3 mice. Inset shows example traces of NMDAR-mediated EPSCs before (control), in the presence of 3 μM ifenprodil, and 20 min after ethanol application in the presence of ifenprodil. For clarity, the stimulus artifacts on the EPSC traces have been removed. (g) NMDAR-mediated EPSCs measured before and during co-application of 3 μM ifenprodil and 0.4 mM NVP-AAM077 in 1 min bins. n=5 from 3 mice. (h) Decay of NMDAR EPSCs measured after stable application of either ifenprodil (3 μM; n=7, patterned bar) or NVP-44AM077 (0.4 μM; n=6, black bar) and their respective baselines (white bars). *p<0.05, paired t-test comparing change in decay time induced by each drug to their respective baselines.

FIG. 6. Following 2 weeks of ethanol self-administration, the effects of Tat-H-ras dominant negative and the vehicle were tested following systemic injection at 15:00 hours; ethanol and water intake were measured 18 hours later at 09:00 hours. Treatments were tested using a within-subjects design; subjects received two injections per week, with each animal receiving each dose in a counterbalanced fashion. Injection volumes were 1-3 ml per 100 g body weight.

### DETAILED DESCRIPTION

The present invention relates to the discovery that modulators of H-ras modulate symptoms of maladaptive substance use. Accordingly, the invention provides a method of mitigating a symptom of maladaptive substance use in a subject, as well as a pharmaceutical composition useful in treating maladaptive substance use. The invention also includes various screening methods based on assaying test agents for their ability to bind to and/or antagonize H-ras. Another aspect of the invention is a diagnostic method for assessing a subject's risk for maladaptive substance use. These methods are of particular interest with respect to common drugs of abuse, such as opioids, psychostimulants, cannabinoids, empathogens, dissociative drugs, and ethanol.

Definitions

Terms used in the claims and specification are defined as set forth below unless otherwise specified.

The following terms encompass polypeptides that are identified in Genbank by the following designations, as well as polypeptides that are at least about 70% identical to polypeptides identified in Genbank by these designations: H-ras. In alternative embodiments, these terms encompass polypeptides identified in Genbank by these designations and sharing at least about 80, 90, 95, 96, 97, 98, or 99% identity.

A “modulator” of a polypeptide is either an inhibitor or an enhancer of polypeptide function.

A modulator “acts directly on” a polypeptide when the modulator binds to the polypeptide.

A modulator “acts indirectly on” a polypeptide when the modulator binds to a molecule other than the polypeptide, which binding results in modulation of polypeptide function.

An “inhibitor” or “antagonist” of a polypeptide is an agent that reduces, by any mechanism, any polypeptide action, as compared to that observed in the absence (or presence of a smaller amount) of the agent. An inhibitor of a polypeptide can affect: (1) the expression, mRNA stability, protein trafficking, modification (e.g., farnesylation), or degradation of a polypeptide, or (2) one or more of the normal functions of the polypeptide. An inhibitor of a polypeptide can be non-selective or selective. Preferred inhibitors (antagonists) are generally small molecules that act directly on, and are selective for, the target polypeptide.

An exemplary inhibitor of H-ras is: L-739,749 (Xu, Y., et al. (2002) J. Neuroscience 22:9194-9202). Additional H-ras inhibitors useful in the invention are described in Bell, I. (2004) J. Med. Chem. 47:1869-78 and Bruner, B. (2003) Cancer Res. 63:5656-68.

An “enhancer” or “agonist” is an agent that increases, by any mechanism, any polypeptide action, as compared to that observed in the absence (or presence of a smaller amount) of the agent. An enhancer of a polypeptide can affect: (1) the expression, mRNA stability, protein trafficking, modification (e.g., farnesylation), or degradation of a polypeptide, or (2) one or more of the normal functions of the polypeptide. An enhancer of a polypeptide can be non-selective or selective. Preferred enhancers (agonists) are generally small molecules that act directly on, and are selective for, the target polypeptide.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

The phrases “an effective amount” and “an amount sufficient to” refer to amounts of a biologically active agent that produce an intended biological activity.

The term “maladaptive substance use” refers to the use of any substance that results in adverse consequences for the user that outweigh any benefits derived from the substance. Substances that are use in a maladaptive manner are generally consumed or administered (usually self-administered) to the body, by any route of administration, to produce an effect on the body that the user generally experiences as pleasurable. The substance can be a single substance (cocaine, for example) or a type of substance (e.g., food, in general). The adverse consequences can include, for example, adverse effects on health, the ability to care for oneself, the ability to form and maintain human relationships, and/or the ability to work. The adverse consequences are generally significant enough that the user would like to control, reduce, or end substance use or, alternatively, the user's family members and/or friends would like to see the user control, reduce, or end substance use. Maladaptive substance use includes uncontrollable craving for the substance; substance dependence, including psychological and/or physical dependence; and substance abuse; as well as any of the individual symptoms of substance dependence and/or abuse listed bellow.

A “symptom of maladaptive substance use” includes any symptom arising from maladaptive substance use. Thus, a symptom of maladaptive substance use arises from the previous, and/or ongoing, use of a substance. Examples include, but are not limited to, elevated drug reward, incentive salience for the drug, drug craving, drug seeking, and drug consumption, as compared to that in a normal population (i.e., one that is not using the substance in a maladaptive manner), as well as any of the individual symptoms of substance dependence and/or abuse listed below.

“Substance dependence” includes a maladaptive pattern of substance use, leading to clinically significant impairment or distress, as manifested by three (or more) of the following symptoms, occurring at any time in the same 12-month period:

(1) Tolerance, as defined by either of the following: (a) a need for markedly increased amounts of the substance to achieve intoxication or desired effect, or (b) markedly diminished effect with continued use of the same amount of the substance;

(2) Withdrawal, as manifested by either of the following: (a) the characteristic withdrawal syndrome for the substance, or (b) the same (or closely related) substance is taken to relieve or avoid withdrawal symptoms;

(3) The substance is often taken in larger amounts or over a longer period than was intended;

(4) There is a persistent desire or unsuccessful efforts to cut down or control substance use;

(5) A great deal of time is spent in activities necessary to obtain the substance (e.g., visiting multiple doctors or driving long distances), use the substance (e.g., chain-smoking), or recover from its effects;

(6) Important social, occupational, or recreational activities are given up or reduced because of substance use; and

(7) The substance use is continued despite knowledge of having a persistent or recurrent physical or psychological problem that is likely to have been caused or exacerbated by the substance (e.g., current cocaine use despite recognition of cocaine-induced depression, or continued drinking despite recognition that an ulcer was made worse by alcohol consumption). (See American Psychiatric Association, Diagnostic Criteria for DSM-IV, Washington D.C., APA, 1994.)

A person is “dependent upon a substance” if such person is determined by a licensed physician or other appropriate accredited medical personnel to meet the criteria for substance dependence with respect to such substance.

“Substance abuse” includes a maladaptive pattern of substance use leading to clinically significant impairment or distress, as manifested by one (or more) of the following, occurring within a 12-month period:

(1) recurrent substance use resulting in a failure to fulfill major role obligations at work, school, or home (e.g., repeated absences or poor work performance related to substance use; substance-related absences, suspensions, or expulsions from school; neglect of children or household);

(2) recurrent substance use in situations in which it is physically hazardous (e.g., driving an automobile or operating a machine when impaired by substance use);

(3) recurrent substance-related legal problems (e.g., arrests for substance-related disorderly conduct); and

(4) continued substance use despite having persistent or recurrent social or interpersonal problems caused or exacerbated by the effects of the substance (e.g., arguments with spouse about consequences of intoxication, physical fights). (See American Psychiatric Association, Diagnostic Criteria for DSM-IV, Washington D.C., APA, 1994.)

A person is “an abuser of a substance” or “abusive of a substance” if such person is determined by a licensed physician or other appropriate accredited medical personnel to meet the criteria for substance abuse with respect to such substance.

The terms drug reward, incentive salience for the drug, drug craving, drug seeking, and drug consumption refer to “drugs” because these concepts have generally been used in the drug dependence/abuse context. However, it should be understood that these terms, as used herein, also encompass reward, incentive salience, craving, seeking and consumption of any substance that is used in a maladaptive manner.

The term “drug reward” refers to the tendency of a drug or substance to cause pleasurable effects which induce a subject to alter their behavior to obtain more of the drug or substance.

The phrase “incentive salience for the drug” refers to a particular form of motivation to consume a previously experienced drug or substance that results from a hypersensitive neural state thought to be mediated by dopaminergic systems.

The term “drug craving” refers to the desire to experience the effects of a previously experienced drug or substance or to ameliorate the negative symptoms of drug or substance withdrawal by taking more of a previously experienced drug or substance.

The term “drug seeking” refers to behavior aimed at obtaining a drug or substance, even in the face of negative health and social consequences. Drug seeking is often uncontrollable and compulsive.

“Drug consumption” refers to the amount of drug or substance consumed by a subject over a selected period of time.

A “drug of abuse” includes any substance, the excessive consumption or administration of which can result in a diagnosis of substance dependence or abuse as defined herein or as defined by the current DSM Criteria promulgated by the American Psychiatric Association or equivalent criteria. Drugs of abuse include, without limitation, an opioid, a psychostimulant, a cannabinoid, an empathogen, a dissociative drug, and ethanol. Thus, for example, heroin, cocaine, methamphetamines, cannabis, 3-4 methylenedioxy-methamphetamine (MDMA), barbiturates, phencyclidine (PCP), ketamine, and ethanol are all drugs of abuse, as defined herein.

A “test agent” is any agent that can be screened in the prescreening or screening assays of the invention. The test agent can be any suitable composition, including a small molecule, peptide, or polypeptide.

An agent is said to “modulate” a symptom of maladaptive substance use if the agent inhibits (i.e., reduces or prevents) or enhances (i.e., increases) the symptom.

An agent is said to “mitigate” a symptom of maladaptive substance use if the agent inhibits (i.e., reduces or prevents) the symptom.

The term “therapy,” as used herein, encompasses the treatment of an existing condition as well as preventative treatment (i.e., prophylaxis). Accordingly, “therapeutic” effects and applications include prophylactic effects and applications, respectively.

A used herein, the term “high risk” refers to an elevated risk as compared to that of an appropriate matched (e.g., for age, sex, etc.) control population.

Method of Mitigating a Symptom of Maladaptive Substance Use

A. In General

The invention provides a method of mitigating a symptom of maladaptive substance use. The method entails inhibiting a Ras protein, such as H-ras, in a subject, whereby the symptom of maladaptive substance use is reduced or prevented. Generally, the method is carried out by administering an effective amount of a Ras (e.g., H-ras) inhibitor to a subject. The invention encompasses methods applicable to the maladaptive use of any substance, including any substance that is used maladaptively that is described individually herein, any grouping of individually described substances, any generic group of substances described herein, and any generic group of substances described herein, with the proviso that one or more of the individually described substances is excluded from the generic group. In particular, the method is useful for addressing undesirable effects or behaviors associated with a variety of drugs of abuse, as well as those associated with other substances, such as food, particular types of food (e.g., sugar, caffeine), and nicotine. In one embodiment, the method is employed to treat maladaptive use of any substance that is used maladaptively, except food. In another embodiment, the method is employed to treat maladaptive use of any substance that it used maladaptively except food and nicotine. In yet another embodiment, the method is employed to treat maladaptive use of a drug of abuse.

In particular embodiments, the method is used to reduce or prevent symptoms associated with drugs such as opioids, psychostimulants, cannabinoids, empathogens, alcohol, and the like. Exemplary opioids include morphine, codeine, heroin, butorphanol, hydrocodone, hydromorphone, levorphanol, meperidine, nalbuphine, oxycodone, fentanyl, methadone, propoxyphene, remifentanil, sufentanil, and pentazocine. Psychostimulants include drugs that stimulate the central nervous system, such as, for example, amphetamine, cocaine, methamphetamine, methylphenidate (ritalin), and methylene dioxy-methamphetamine (MDMA). Exemplary cannabinioids include tetrahydrocannabinol (THC), dronabinol, and arachidonylethanolamide (anandamide, AEA). Empathogens include phenethylamines, such as, for example, MDMA, 3,4-methylenedioxy amphetamine (MDA), 3,4-methylenedioxy-N-ethylamphetamine (MDEA), 2,5-Dimethoxy-4-iodo-phenethylamine or 1-(2,5-dimethoxy-4-iodophenyl)-2-aminoethane (2C—I), 2,5-dimethoxy-4-bromo-phenethylamine (2C—B), and N-methyl-1-(3,4-methylenedioxyphenyl)-2-butanamine. Dissociative drugs include PCP and ketamine.

Examples of symptoms of maladaptive substance use that can be mitigated according to the method of the invention include elevated: drug reward, incentive salience for a drug, drug craving, drug seeking, and drug consumption.

The subject of the method can be any individual that expresses H-ras. Examples of suitable subjects include research animals, such as mice, rats, guinea pigs, rabbits, cats, dogs, as well as monkeys and other primates, and humans. The subject can be an individual who is regularly, or intermittently, using a substance in a maladaptive manner or an individual who is at risk for such use. In particular embodiments, the subject uncontrollably craves, is physically or psychologically dependent upon, or an abuser of, a drug of abuse. The method finds particular application in treating subjects in whom craving, physical or psychological drug dependence, and/or abuse has recently been identified, (e.g., as part of a drug rehabilitation program) or who have been found through genetic testing to be at risk.

The method of the invention entails inhibiting H-ras to a degree sufficient to reduce or prevent one or more symptom(s) of maladaptive substance use. In various embodiments, H-ras is inhibited by at least about 10, 20, 30, 40, 50, 60, 70, 80, 90, and 95 percent, as determined by any suitable measure of H-ras inhibition (such as, for example, any of the assays described herein).

1. Ras Inhibitors

Any kind of H-ras inhibitor that is tolerated by the subject can be employed in the method of the invention. Thus, the inhibitor can be a polypeptide (such as, e.g., an anti-H-ras antibody), a polynucleotide (e.g., one that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit H-ras.

Inhibition of H-ras can be achieved by any available means, e.g., by modulating: (1) the expression, mRNA stability, protein trafficking, modification (e.g., farnesylation), or degradation of H-ras, or (2) one or more of the normal functions of H-ras.

Under normal circumstances, Ras proteins cycle between an active (GTP-bound) state and an inactive (GDP-bound) state. Ras activation occurs by exchange of bound GDP for GTP, which is facilitated by a family of guanine nucleotide exchange factors. Ras inactivation occurs by hydrolysis of bound GTP to GDP. This reaction is facilitated by GTPase activating proteins (GAPs). Thus, H-ras can be inhibited by blocking or reducing guanine nucleotide exchange, e.g., by inhibiting a ras-guanine nucleotide exchange factor (ras-GEF). For example, the dominant negative form of H-ras described in Example 1 (H-Ras DN) sequesters the guanine nucleotide exchange factors necessary for Ras activation and thereby inhibits the activation of endogenous H-ras. See also, Feig, L. (June 1999) Nature Cell Biol. 1 (cellbio.nature.com) for a general description of dominant-inhibitory mutants of Ras proteins. H-ras can also be inhibited by stimulating H-ras' intrinsic GTPase activity, e.g., by stimulating a ras-GTPase activating protein (ras-GAP). An exemplary ras-GAP that can targeted in this embodiment is SynGAP, a neuronal ras-GAP that is selectively expressed in brain and highly enriched at excitatory synapses, where it negatively regulates Ras activity and its downstream signaling pathways. See J Neurosci (Online) (2003 Feb. 15).

Farnesyl protein transferase (FPT) inhibitors represent a leading approach for blocking the function of Ras proteins. FPT catalyzes the addition of an isoprenyl lipid moiety onto a cysteine residue present near the carboxy-terminus of the Ras protein. This is the first step in a post-translational processing pathway that is essential for both Ras membrane-association and Ras-induced oncogenic transformation. A number of FPT inhibitors have been reported, including a variety of peptidomimetic inhibitors as well as other small molecule inhibitors, notably the tricyclic FPT inhibitors exemplified by SCH 66336 (see U.S. Pat. No. 6,777,415).

Classes of compounds that can be used as FPT inhibitors include: fused-ringed tricyclic benzocycloheptapyridines, oligopeptides, peptido-mimetic compounds, farnesylated peptido-mimetic compounds, carbonyl piperazinyl compounds, carbonyl piperidinyl compounds, farnesyl derivatives, and natural products and derivatives.

Examples of compounds that are FPT inhibitors and publications directed to such compounds are as follows: Fused-ring tricyclic benzocycloheptapyridines: WO 95/10514; WO 95/10515; WO 95/10516; WO 96/30363; WO 96/30018; WO 96/30017; WO 96/30362; WO 96/31111; WO 96/31478; WO 96/31477; WO 96/31505; WO 97/23478; International Patent Application No. PCT/US97/17314 (WO 98/15556); International Patent Application No. PCT/US97/15899 (WO 98/11092); International Patent Application No. PCT/US97/15900 (WO 98/11096); International Patent Application No. PCT/US97/15801 (WO 98/11106); International Patent Application No. PCT/US97/15902 (WO 98/11097); International Patent Application No. PCT/US97/15903 (WO 98/11098); International Patent Application No. PCT/US97/15904; International Patent Application No. PCT/US97/15905 (WO 98/11099); International Patent Application No. PCT/US97/15906 (WO 98/11100); International Patent Application No. PCT/US97/15907 (WO 98/11093); International Patent Application No. PCT/US97/19976 (WO 98/11091); U.S. application Ser. No. 08/877,049 now abandoned; U.S. application Ser. No. 08/877,366 now U.S. Pat. No. 5,939,416; U.S. application Ser. No. 08/877,399, now U.S. Pat. No. 5,852,034; U.S. application Ser. No. 08/877,336, now U.S. Pat. No. 5,877,177; U.S. application Ser. No. 08/877,269 now abandoned; U.S. application Ser. No. 08/877,050 now abandoned; U.S. application Ser. No. 08/877,052 now abandoned; U.S. application Ser. No. 08/877,051 now abandoned; U.S. application Ser. No. 08/877,498 now abandoned; U.S. application Ser. No. 08/877,057 now abandoned; U.S. application Ser. No. 08/877,739 now abandoned; U.S. application Ser. No. 08/877,677 now U.S. Pat. No. 5,925,639; U.S. application Ser. No. 08/877,741 now abandoned; U.S. application Ser. No. 08/877,743 now abandoned; U.S. application Ser. No. 08/877,457 now abandoned; U.S. application Ser. No. 08/877,673 now abandoned; U.S. application Ser. No. 08/876,507 now abandoned; and U.S. application Ser. No. 09/216,398. See also U.S. Pat. No. 6,777,415. Each of these publications is incorporated herein by reference in its entirety.

Some FPT inhibitors are oligopeptides, especially tetrapeptides, or derivatives thereof, based on the formula Cys-Xaa₁-Xaa₂-Xaa₃, where Xaa₃ represents a serine, methionine or glutamine residue, and Xaa₁ and Xaa₂ can represent a wide variety of amino acid residues, but especially those with an aliphatic side-chain. Their derivatives may or may not have three peptide bonds; thus, it has been found that reduction of a peptide bond —CO—NH— to a secondary amine grouping, or even replacement of the nitrogen atoms in the peptide chain with carbon atoms (provided that certain factors such as general shape of the molecule and separation of the ends are largely conserved) affords compounds that are frequently more stable than the oligopeptides and, if active, have longer activity. Such compounds are referred to herein as peptido-mimetic compounds. Oligopeptides (mostly tetrapeptides but also pentapeptides) including the formula Cys-Xaa₁-Xaa₂-Xaa₃ are described, for example, in EPA 461,489; EPA 520,823; EPA 528,486; and WO 95/11917. Peptido-mimetic compounds, especially Cys-Xaa-Xaa-Xaa-mimetics, are described in EPA 535,730; EPA 535,731; EPA 618,221; WO 94/09766; WO 94/10138; WO 94/07966; U.S. Pat. Nos. 5,326,773; 5,340,828; 5,420,245; WO 95/20396; U.S. Pat. No. 5,439,918; and WO 95/20396. Farnesylated peptido-mimetic compounds, especifically farnesylated Cys-Xaa-Xaa-Xaa-mimetics, are described in GB-A 2,276,618. Other peptido-mimetic compounds are described in U.S. Pat. No. 5,352,705; WO 94/00419; WO 95/00497; WO 95/09000; WO 95/09001; WO 95/12612; WO 95/25086; EPA 675,112; and FR-A 2,718,149. Each of these publications is incorporated herein by reference in its entirety.

Farnesyl derivatives that inhibit FPT are found in EPA 534,546; WO 94/19357; WO 95/08546; EPA 537,007; and WO 95/13059. FPT inhibitors that are natural products or derivatives thereof are described in WO 94/18157; U.S. Pat. No. 5,430,055; GB-A 2,261,373; GB-A 2,261,374; GB-A 2,261,375; U.S. Pat. Nos. 5,420,334; 5,436,263. Each of these publications is incorporated herein by reference in its entirety.

Other FPT inhibitory compounds are found in WO 94/26723; WO 95/08542; U.S. Pat. No. 5,420,157; WO 95/21815; WO 96/31501; WO 97/16443; WO 97/21701; U.S. Pat. Nos. 5,578,629; 5,627,202; WO 96/39137; WO 97/18813; WO 97/27752WO 97/27852; WO 97/27853; WO 97/27854; WO 97/36587; WO 97/36901; WO 97/36900; WO 97/36898; WO 97/36897; WO 97/36896; WO 97/36892; WO 97/36891; WO 97/36890; WO 97/36889; WO 97/36888; WO 97/36886; WO 97/36881; WO 97/36879; WO 97/36877; WO 97/36876; WO 97/36875; WO 97/36605; WO 97/36593; WO 97/36592; WO 97/36591; WO 97/36585; WO 97/36584; WO 97/36583; U.S. Pat. No. 6,277,871; U.S. Pat. No. 6,248,919; U.S. Pat. No. 5,831,115; U.S. Pat. No. 5,783,593; U.S. Pat. No. 5,631,401. Each of these publications is incorporated herein by reference in its entirety.

Exemplary FTP inhibitors suitable for use in the invention, include R115777 (tipifamib, Zarnestra™, Johnson & Johnson, NJ, USA) and derivatives thereof; SCH66336 (Ionafarnib, Sarasar™, Schering-Plough, NJ, USA) and derivatives thereof; as well as ABT-100 (Abbott Laboratories, IL, USA; see, e.g., Ferguson, D. et al., Clinical Cancer Research (2005) Vol. 11, 3045-3054 [incorporated herein by reference in its entirety]).

Reviews of FPT inhibitors useful in the invention include: Bell, I. (2004) J. Med. Chem. 47:1869-78; Bruner, B. (2003) Cancer Res. 63:5656-68; and Graham (1995) Exp. Opin. Ther. Patents 5: 1269-1285. An exemplary FPT inhibitor of H-ras is: L-739,749 (Xu, Y., et al. (2002) J Neuroscience 22:9194-9202). Each of these publications is incorporated herein by reference in its entirety.

H-ras inhibitor can be non-selective or selective for H-ras. Preferred embodiments employ a selective H-ras antagonist.

In certain embodiments, the H-ras inhibitor can be, e.g., a peptide or a small molecule identified through a screening assay of the invention, which are described below.

In other embodiments, H-ras inhibition is achieved by reducing the level of H-ras polypeptides in the cells or inhibiting H-ras function by various means that entail introducing polynucleotide inhibitors into cells. H-ras levels can be reduced using, e.g., antisense, catalytic RNA/DNA, RNA interference (RNAi), or “knock-out” techniques. H-ras expression/function can also be inhibited using intrabodies.

a. Antisense Methods

H-ras gene expression can be reduced or entirely blocked by the use of antisense molecules. An “antisense sequence or antisense polynucleotide” is a polynucleotide that is complementary to the H-ras coding mRNA sequence or a subsequence thereof. Binding of the antisense molecule to the H-ras mRNA interferes with normal translation of the H-ras polypeptide.

Thus, in particular embodiments, the invention provides antisense molecules useful for inhibiting H-ras. Suitable antisense molecules include oligonucleotides and oligonucleotide analogs that are hybridizable with H-ras mRNA. The oligonucleotides and oligonucleotide analogs are able to inhibit the function of the RNA, either its translation into protein, its translocation into the cytoplasm, or any other activity necessary to its overall biological function. The failure of the mRNA to perform all or part of its normal functions results in a partial or complete inhibition of expression of H-ras polypeptides.

Oligonucleotides useful in the antisense methods of the invention include polynucleotides formed from naturally-occurring bases and/or cyclofuranosyl groups joined by native phosphodiester bonds. The term “oligonucleotide” encompasses moieties that function similarly to oligonucleotides, but that have non-naturally occurring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short-chain alkyl or cycloalkyl structures. In accordance with other preferred embodiments, the phosphodiester bonds are substituted with structures that are, at once, substantially non-ionic and non-chiral, or with structures which are chiral and enantiomerically specific. Persons of ordinary skill in the art will be able to select other linkages for use in the practice of the invention.

In an exemplary embodiment, the internucleotide phosphodiester linkage is replaced with a peptide linkage. Such peptide polynucleotides tend to show improved stability, penetrate the cell more easily, and show enhanced affinity for their target. Methods of making peptide polynucleotides are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides useful in the antisense methods of the invention may also include one or more modified base forms. Thus, purines and pyrimidines other than those normally found in nature may be employed. Similarly, the furanosyl portions of the nucleotide subunits may also be modified, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are: OH, SH, SCH₃, F, OCH₃, OCN, O(CH₂)[n]NH₂ or O(CH₂)[n]CH₃, where n is from 1 to about 10, and other substituents having similar properties.

All such analogs can be used in the antisense methods of the invention so long as the analogs function effectively to hybridize with H-ras mRNA and inhibit the function of that RNA.

Antisense oligonucleotides in accordance with this invention preferably comprise from about 3 to about 50 subunits (i.e., bases in unmodified polynucleotides). It is more preferred that such oligonucleotides and analogs comprise from about 8 to about 25 subunits and still more preferred to have from about 12 to about 20 subunits. The oligonucleotides used in accordance with this invention can be conveniently and routinely made through the well-known technique of solid phase synthesis. Equipment for such synthesis is sold by several vendors (e.g. Applied Biosystems).

Antisense oligonucleotides of the invention can be synthesized, formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotides. Those of skill in the art will readily appreciate that this discussion is equally applicable to antisense oligonucleotides, catalytic RNAs and DNAs, and double-stranded RNAs used in RNAi.

b. Catalytic RNAs and DNAs

(1) Ribozymes

In another approach, H-ras expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (H-ras) RNA, preferably at greater than stoichiometric concentration. The ribozymes of the invention typically consist of RNA, but such ribozymes may also be composed of polynucleotide molecules comprising chimeric polynucleotide sequences (such as DNA/RNA sequences) and/or polynucleotide analogs (e.g., phosphorothioates).

Accordingly, one aspect of the present invention includes ribozymes have the ability to inhibit H-ras expression. Such ribozymes may, e.g., be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585); Rossi et al. (1991) Pharmac. Ther. 50: 245-254) or a “hairpin” (see, e.g., U.S. Pat. No. 5,254,678 and Hampel et al., European Patent Publication No. 0 360 257, published Mar. 26, 1990; Hampel et al. (1990) Nucl. Acids Res. 18: 299-304), and have the ability to specifically target and cleave and H-ras polynucleotides.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:1). Suitable H-ras recognition or target sequences for hairpin ribozymes can be readily determined from the H-ras sequence.

The sequence requirement at the cleavage site for the hammerhead ribozyme is any RNA sequence consisting of NUX (where N is any of G, U, C, or A and X represents C, U, or A). Accordingly, the same target within the hairpin leader sequence, GUC, is useful for the hammerhead ribozyme. The additional nucleotides of the hammerhead ribozyme or hairpin ribozyme are determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an 8-base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. Preferred ribozymes of the invention hybridize efficiently to target sequences at physiological temperatures, making them particularly well suited for use in vivo.

Ribozymes, as well as DNA encoding such ribozymes, and other suitable polynucleotide molecules can be chemically synthesized using methods well known in the art for the synthesis of polynucleotide molecules. Alternatively, Promega, Madison, Wis., USA, provides a series of protocols suitable for the production of RNA molecules such as ribozymes. The ribozymes also can be prepared from a DNA molecule or other polynucleotide molecule (which, upon transcription, yields an RNA molecule) operably linked to an RNA polymerase promoter, e.g., the promoter for T7 RNA polymerase or SP6 RNA polymerase (e.g., a vector that provides an initiation site and template for transcription). Accordingly, also provided by this invention are polynucleotide molecules, e.g., DNA or cDNA, coding for the ribozymes of this invention. When the vector also contains an RNA polymerase promoter operably linked to the polynucleotide molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J. 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the corresponding phosphothio analog for use in liposome delivery systems. This modification also renders the ribozyme resistant to endonuclease activity.

Ribozymes, or polynucleotides encoding them (e.g., DNA vectors) can be formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotides.

By employing a vector containing an encoded ribozyme linked to a promoter for RNA transcription, the RNA can be produced in the host cell when the host cell is grown under suitable conditions favoring transcription of the vector. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J. Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing such vectors, the nucleotide sequence endoding the ribozyme is preferably operably linked to a strong promoter such as, e.g., the lac, SV40 late, SV40 early, or lambda promoters.

(2) Catalytic DNA

In a manner analogous to ribozymes, DNA molecules are also capable of catalytic (e.g. nuclease) activity. For example, highly catalytic species have been developed by directed evolution and selection. Beginning with a population of 10¹⁴ DNAs containing 50 random nucleotides, successive rounds of selective amplification enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ was obtained (see, e.g., Breaker and Joyce (1994) Chem Biol 4: 223-229).

In later work, using a similar strategy, a DNA enzyme was made that could cleave almost any targeted RNA substrate under simulated physiological conditions. The enzyme is composed of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹min⁻¹ under multiple turnover conditions, exceeding that of any other known polynucleotide enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to H-ras mRNA and can be used in essentially the same manner as described above for H-ras ribozymes.

c. RNAi Methods

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. This approach is based on the observation that injection of antisense or sense RNA strands into C. elegans cells resulted in gene-specific inactivation (Guo and Kempheus (1995) Cell 81: 611-620). While gene inactivation by the antisense strand was expected, gene silencing by the sense strand was unexpected. Surprisingly, it was determined that the gene-specific inactivation was actually due to trace amounts of contaminating dsRNA (Fire et al. (1998) Nature 391: 806-811).

Since then, this mode of post-transcriptional gene silencing has been demonstrated in a wide variety of organisms: plants, flies, trypanosomes, planaria, hydra, zebrafish, and mice (Zamore et al. (2000) Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expression, which do not allow for gene silencing during various stages of the developmental process (Gura (2000) Nature 404:804-808).

dsRNA can be formulated, and administered to cells, tissues, or organisms in accordance with standard practice. General considerations with respect to administration and dose are discussed below, as are formulations containing at least one component that facilitates entry of a polynucleotide into a cell (discussed below with respect to compositions containing polynucleotides). Additionally, dsRNA can be synthesized using one or more vectors designed to transcribe the two complementary RNA strands that hybridize to form the dsRNA (see the discussion of this approach with respect to ribozymes, above). These may be introduced into host cells using any of the techniques described herein or known in the art for this purpose.

After introduction into cells, it has been shown that dsRNA is cleaved by a nuclease into 21-23-nucleotide fragments. These fragments, in turn, target the homologous region of their corresponding mRNA, hybridize, and result in a double-stranded substrate for a nuclease that degrades it into fragments of the same size (Hammond et al. (2000) Nature 404:293-298; Zamore et al. (2000) Cell 101:25-33).

d. “Knock-Out” Methods

In another approach, H-ras can be inhibited simply by “knocking out” the H-ras gene. Typically, this is accomplished by disrupting the H-ras gene, the promoter regulating the gene or sequences between the promoter and the gene. Such disruption can be specifically directed to H-ras by homologous recombination where a “knockout construct” contains flanking sequences complementary to the domain to which the construct is targeted. Insertion of the knockout construct (e.g., into the H-ras gene) results in disruption of that gene. The phrases “disruption of the gene” and “gene disruption” refer to insertion of a nucleic acid sequence into one region of the native DNA sequence (usually one or more exons) and/or the promoter region of a gene so as to reduce or prevent expression of that gene in the cell, as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The genomic DNA sequence to be used in producing the knockout construct is digested with a particular restriction enzyme selected to cut at a location(s) such that a new DNA sequence encoding, e.g., a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable; however, typically it is an antibiotic resistance gene or other gene whose expression or presence in the genome can easily be detected. The marker gene is usually operably linked to its own promoter or to another strong promoter from any source that will be active, or can easily be activated, in the cell into which it is introduced; however, the marker gene need not be linked to its own promoter as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to, neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement).

The resulting knockout constructs can be delivered to cells in vivo using gene therapy delivery vehicles (e.g., retroviruses, liposomes, lipids, dendrimers, etc.). Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Although embryonic stem (ES) cells can be employed to produce knockout animals, ES cells are not required. In various embodiments, knockout animals can be produced using methods of somatic cell nuclear transfer. In preferred embodiments using such an approach, a somatic cell is obtained from the species in which the H-ras gene is to be knocked out. The cell is transfected with a construct that introduces a disruption in the H-ras gene (e.g., via homologous recombination). Cells harboring a knocked out H-ras gene are selected, e.g., by selecting for expression of a marker encoded by a marker gene used to disrupt the native gene. The nucleus of cells harboring the knockout is then placed in an unfertilized enucleated egg (e.g., eggs from which the natural nuclei have been removed by microsurgery). Once the transfer is complete, the recipient eggs contain a complete set of genes, just as they would if they had been fertilized by sperm. The eggs are then cultured for a period before being implanted into a host mammal (of the same species that provided the egg) where they are carried to term, culminating in the birth of a transgenic animal comprising a nucleic acid construct containing one or more disrupted H-ras genes.

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Somatic cell nuclear transfer simplifies transgenic procedures by employing a differentiated cell source that can be clonally propagated. This eliminates the need to maintain the cells in an undifferentiated state, thus, genetic modifications, both random integration and gene targeting, are more easily accomplished. Also, by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report. Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

e. Intrabodies

In still another embodiment, H-ras expression/activity can be inhibited by introducing a nucleic acid construct that expresses an intrabody into the target cells. An intrabody is an intracellular antibody, in this case, capable of recognizing and binding to an H-ras polypeptide. The intrabody is expressed by an “antibody cassette” containing: (1) a sufficient number of nucleotides encoding the portion of an antibody capable of binding to the target (H-ras polypeptide) operably linked to (2) a promoter that will permit expression of the antibody in the cell(s) of interest. The construct encoding the intrabody is delivered to the cell where the antibody is expressed intracellularly and binds to the target H-ras, thereby disrupting the target from its normal action.

In a preferred embodiment, the “intrabody gene” of the antibody cassette includes a cDNA encoding heavy chain variable (V_{H}) and light chain variable (V_{L}) domains of an antibody which can be connected at the DNA level by an appropriate oligonucleotide linker, which on translation, forms a single peptide (referred to as a single chain variable fragment, “sFv”) capable of binding to a target such as an H-ras protein. The intrabody gene preferably does not encode an operable secretory sequence, and thus the expressed antibody remains within the cell.

Anti-H-ras antibodies suitable for use/expression as intrabodies in the methods of this invention can be readily produced by a variety of methods. Such methods include, but are not limited to, traditional methods of raising polyclonal antibodies, which can be modified to form single chain antibodies, or screening of, e.g., phage display libraries to select for antibodies showing high specificity and/or avidity for H-ras.

The antibody cassette is delivered to the cell by any means suitable for introducing polynucleotides into cells. A preferred delivery system is described in U.S. Pat. No. 6,004,940. Methods of making and using intrabodies are described in detail in U.S. Pat. Nos. 6,072,036, 6,004,940, and 5,965,371.

B. Compositions

For research and therapeutic applications, a Ras inhibitor, such as an H-ras inhibitor, is generally formulated to deliver inhibitor to a target site in an amount sufficient to inhibit the targeted Ras protein at that site.

Inhibitor compositions of the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Preferred embodiments include sustained-release pharmaceutical compositions. An exemplary sustained-release composition has a semipermeable matrix of a solid hydrophobic polymer to which the inhibitor is attached or in which the inhibitor is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are typically in the form of shaped articles, such as films, or microcapsules.

Where the inhibitor is a polypeptide, exemplary sustained release compositions include the polypeptide attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” protein retains the desired function(s).

In another embodiment, a sustained-release composition includes a liposomally entrapped inhibitor. Liposomes are small vesicles composed of various types of lipids, phospholipids, and/or surfactants. These components are typically arranged in a bilayer formation, similar to the lipid arrangement of biological membranes. Liposomes containing inhibitors are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide inhibitor or a polynucleotide encoding a polypeptide inhibitor of H-ras. Such compositions optionally include other components, as for example, a storage solution, such as a suitable buffer, e.g., a physiological buffer. In a preferred embodiment, the composition is a pharmaceutical composition and the other component is a pharmaceutically acceptable carrier, as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

C. Administration

Pharmaceutical compositions according to the invention are generally administered systemically. Methods for systemic administration do not differ from known methods for administering small-molecule drugs or therapeutic polypeptides, peptides, or polynucleotides them. Suitable routes of administration include, for example, topical, intravenous, intraperitoneal, intracerebral, intraventricular, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

In certain embodiments, the compositions are delivered through the skin using a conventional transdermal drug delivery system, i.e., a transdermal “patch” wherein the composition is typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of a selected composition that is ultimately available for delivery to the surface of the skin. Thus, for example, the reservoir may include the composition in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the reservoir and skin contact adhesive are present as separate and distinct layers, with the adhesive underlying the reservoir which, in this case, may be either a polymeric matrix as described above or a liquid or hydrogel reservoir, or it may take some other form. The backing layer in these laminates, which serves as the upper surface of the device, preferably functions as a primary structural element of the patch and provides the device with much of its flexibility. The material selected for the backing layer is preferably substantially impermeable to the selected composition and any other materials that are present.

Transdermal patches according to the invention can include a rate-limiting patch membrane. The size of the patch and or the rate-limiting membrane can be chosen to deliver the transdermal flux rates desired. A release liner, such as a polyester release liner, can also be provided to cover the adhesive layer prior to application of the patch to the skin as is conventional in the art. This patch assembly can be packaged in an aluminum foil or other suitable pouch, again, as is conventional in the art.

In other embodiments, the compositions of the invention are administered in implantable depot formulations. A wide variety of approaches to designing depot formulations that provide sustained release of an active agent are known and are suitable for use in the invention. Generally, the components of such formulations are biocompatible and may be biodegradable. Biocompatible polymeric materials have been used extensively in therapeutic drug delivery and medical implant applications to effect a localized and sustained release. See Leong et al., “Polymeric Controlled Drug Delivery”, Advanced Drug Delivery Rev., 1: 199-233 (1987); Langer, “New Methods of Drug Delivery”, Science, 249:1527-33 (1990); Chien et al., Novel Drug Delivery Systems (1982). Such delivery systems offer the potential of enhanced therapeutic efficacy and reduced overall toxicity.

If an implant is intended for use as a drug delivery or other controlled-release system, using a biodegradable polymeric carrier is one effective means to deliver the therapeutic agent locally and in a controlled fashion, see Langer et al., “Chemical and Physical Structures of Polymers as Carriers for Controlled Release of Bioactive Agents”, J. Macro. Science, Rev. Macro. Chem. Phys., C23(1), 61-126 (1983). As a result, less total drug is required, and toxic side effects can be minimized. Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists”, Controlled Release of Bioactive Materials, 19-44 (Richard Baker ed., 1980); poly(amino acids) and pseudo-poly(amino acids) (Pulapura et al. “Trends in the Development of Bioresorbable Polymers for Medical Applications”, J. Biomaterials Appl., 6:1, 216-50 (1992); polyurethanes (Bruin et al., “Biodegradable Lysine Diisocyanate-based Poly(Glycolide-co-.epsilon. Caprolactone)-Urethane Network in Artificial Skin”, Biomaterials, 11:4, 291-95 (1990); polyorthoesters (Heller et al., “Release of Norethindrone from Poly(Ortho Esters)”, Polymer Engineering Sci., 21:11, 727-31 (1981); and polyanhydrides (Leong et al., “Polyanhydrides for Controlled Release of Bioactive Agents”, Biomaterials 7:5, 364-71 (1986).

Thus, for example, H-ras inhibitor composition can be incorporated into a biocompatible polymeric composition and formed into the desired shape outside the body. This solid implant is then typically inserted into the body of the subject through an incision. Alternatively, small discrete particles composed of these polymeric compositions can be injected into the body, e.g., using a syringe. In an exemplary embodiment, H-ras inhibitor composition can be encapsulated in microspheres of poly (D,L-lactide) polymer suspended in a diluent of water, mannitol, carboxymethyl-cellulose, and polysorbate 80. The polylactide polymer is gradually metabolized to carbon dioxide and water, releasing H-ras inhibitor into the system.

In yet another approach, depot formulations can be injected via syringe as a liquid polymeric composition. Liquid polymeric compositions useful for biodegradable controlled release drug delivery systems are described, e.g., in U.S. Pat. Nos. 4,938,763; 5,702,716; 5,744,153; 5,990,194; and 5,324,519. After injection in a liquid state or, alternatively, as a solution, the composition coagulates into a solid.

One type of polymeric composition suitable for this application includes a nonreactive thermoplastic polymer or copolymer dissolved in a body fluid-dispersible solvent. This polymeric solution is placed into the body where the polymer congeals or precipitates and solidifies upon the dissipation or diffusion of the solvent into the surrounding body tissues. See, e.g., Dunn et al., U.S. Pat. Nos. 5,278,201; 5,278,202; and 5,340,849 (disclosing a thermoplastic drug delivery system in which a solid, linear-chain, biodegradable polymer or copolymer is dissolved in a solvent to form a liquid solution).

H-ras inhibitor composition can also be adsorbed onto a membrane, such as a silastic membrane, which can be implanted, as described in International Publication No. WO 91/04014.

D. Dose

The dose of inhibitor is sufficient to inhibit the H-ras, preferably without significant toxicity. In particular in vivo embodiments, the amount of H-ras inhibitor is sufficient to mitigate a symptom of maladaptive substance use in a subject. For in vivo applications, the dose of inhibitor depends, for example, upon the therapeutic objectives, the route of administration, and the condition of the subject. Accordingly, it is necessary for the clinician to titer the dosage and modify the route of administration as required to obtain the optimal therapeutic effect. Generally, the clinician begins with a low dose and increases the dosage until the desired therapeutic effect is achieved. Starting doses for a given inhibitor can be extrapolated from in vitro and/or animal data.

Methods of Screening for Agents that Modulate a Symptom of Maladaptive Substance Use

The demonstrated role of H-ras in modulation of substance consumption makes Ras an attractive target for agents that modulate one or more symptoms of maladaptive substance use. Accordingly, the invention provides prescreening and screening methods aimed at identifying such agents. Test agents can be prescreened, for example, based on binding to H-ras or on binding to polynucleotides encoding H-ras. Screening methods of the invention can be carried out by: contacting a test agent with H-ras; determining whether the test agent acts as an agonist or an antagonist of the protein; and if so, selecting the test agent as a potential modulator of maladaptive substance use in a subject. For example, test agents can be screened for effects on the levels of H-ras or polynucleotides encoding H-ras (e.g., H-ras mRNA) or for effects on H-ras function.

The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof.

A. Prescreening Based on Binding to H-ras

The invention provides a prescreening method based on assaying test agents for specific binding to Ras proteins, such as H-ras. Agents that specifically bind to H-ras have the potential to modulate H-ras function and thereby modulate one or more symptoms of maladaptive substance use.

In one embodiment, therefore, a prescreening method of the invention entails contacting a test agent with H-ras. Specific binding of the test agent to H-ras is then determined. If specific binding is detected, the test agent is selected as a potential modulator of a symptom of maladaptive substance use.

Such prescreening is generally most conveniently accomplished with a simple in vitro binding assay. Means of assaying for specific binding of a test agent to a polypeptide are well known to those of skill in the art. In preferred binding assays, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various assay formats are discussed in greater detail below.

B. Prescreening Based on Binding to Polynucleotides Encoding H-ras

The invention also provides a prescreening method based on screening test agents for specific binding to a polynucleotide encoding a Ras protein, such as H-ras. Agents that specifically bind to such polynucleotides have the potential to modulate the expression of the encoded H-ras, and thereby modulate one or more symptoms of maladaptive substance use.

In one embodiment, therefore, a prescreening method of the invention entails contacting a test agent with a polynucleotide encoding H-ras. Specific binding of the test agent to the polynucleotide is then determined. If specific binding is detected, the test agent is selected as a potential modulator of a symptom of maladaptive substance use.

Such prescreening is generally most conveniently accomplished with a simple in vitro binding assay, which are well known to those of skill in the art. In preferred binding assays, the polynucleotide is immobilized and exposed to a test agent (which can be labeled), or alternatively, the test agent(s) are immobilized and exposed to the polynucleotide (which can be labeled). The immobilized species is then washed to remove any unbound material and the bound material is detected. To prescreen large numbers of test agents, high throughput assays are generally preferred. Various assay formats are discussed in greater detail below.

C. Screening Based on Levels of H-ras Polypeptides or Polynucleotides

Test agents, including, for example, those identified in a prescreening assay of the invention can also be screened to determine whether the test agent affects the levels of Ras (e.g., H-ras) polypeptides or polynucleotides (e.g., mRNA). Agents that reduce these levels can potentially reduce one or more symptoms of maladaptive substance use. Conversely, agents that increase these levels can potentially enhance such symptom(s).

Accordingly, the invention provides a method of screening for an agent that modulates a symptom of maladaptive substance use in which a test agent is contacted with a cell that expresses H-ras in the absence of test agent. Preferably, the method is carried out using an in vitro assay. In such assays, the test agent can be contacted with a cell in culture or present in a tissue. Alternatively, the test agent can be contacted with a cell lysate or fraction thereof. The level of H-ras or H-ras polynucleotides (e.g., mRNA) is determined in the presence and absence (or presence of a lower amount) of test agent to identify any test agents that alter the level. If the level is altered, the test agent is selected as a potential modulator of a symptom of maladaptive substance use. In a preferred embodiment, an agent that reduces H-ras polypeptide or polynucleotide level is selected as a potential inhibitor of one or more symptoms of maladaptive substance use.

Cells or tissues useful in this screening method include those from any of the species described above in connection with the method of mitigating a symptom of maladaptive substance use. Cells that naturally express H-ras are typically, although not necessarily, employed in this screening method. Alternatively, cells that have been engineered to express H-ras can be used in the method.

1. Sample

As noted above, screening assays are generally carried out in vitro, for example, in cultured cells, in a biological sample (e.g., brain), or fractions thereof. For ease of description, cell cultures, biological samples, and fractions are referred to as “samples” below. The sample is generally derived from an animal (e.g., any of the research animals mentioned above), preferably from a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

2. Polypeptide-Based Assays

H-ras can be detected and quantified by any of a number of methods well known to those of skill in the art. Examples of analytic biochemical methods suitable for detecting H-ras include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one embodiment, H-ras polypeptides are detected/quantified using a ligand binding assay, such as, for example, a radioligand binding assay. Briefly, a sample from a tissue expressing H-ras is incubated with a suitable ligand under conditions designed to provide a saturating concentration of ligand over the incubation period. After ligand treatment, the sample is assayed for radioligand binding. Any ligand that binds to H-ras can be employed in the assay, although H-ras-selective ligands are preferred. Any of H-ras inhibitors discussed above can, for example, be labeled and used in this assay.

In another embodiment, H-ras polypeptide are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of H-ras in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In a preferred embodiment, H-ras polypeptides are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case H-ras). In preferred embodiments, the capture agent is an antibody.

Immunoassays also typically utilize a labeling agent to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the labeling agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled H-ras is added to the sample, and the sample is then contacted with a capture agent. The amount of labeled H-ras bound to the antibody is inversely proportional to the concentration of H-ras present in the sample.

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In particular embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen, or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Antibodies useful in these immunoassays include polyclonal and monoclonal antibodies.

3. Polynucleotide-Based Assays

Changes in H-ras expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

i. Amplification-Based Assays

In one embodiment, amplification-based assays can be used to detect, and optionally quantify, a polynucleotide encoding H-ras. In exemplary amplification-based assays, H-ras mRNA in the sample acts as a template in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of H-ras mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

ii. Hybridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of H-ras mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the H-ras mRNA of interest, and standard methods (such as, e.g., densitometry where the nucleic acid probe is radioactively labeled) can be used to quantify the level of the H-ras mRNA.)

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g., by hand using a pipette) different nucleic acids at different locations on a solid support (e.g., a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a preferred embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can also be carried out using a MicroElectroMechanical System (MEMS), such as the Protiveris' multicantilever array.

iii. Polynucleotide Detection

H-ras polynucleotides can be detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000, 7700, or 7900 (TaqMan®) from Applied Biosystems or the LightCycler® System from Roche.

D. Screening Based on H-ras Function

The invention also provides a screening method based on determining the effect, if any, of a test agent on the level of Ras (e.g., H-ras) function. H-ras function can be assayed by measuring any response mediated by H-ras. Agents that reduce H-ras function can potentially reduce one or more symptoms of maladaptive substance use. Conversely, agents that increase H-ras function can potentially enhance such symptom(s).

Accordingly, the invention provides a method of screening for an agent that inhibits or enhances a symptom of maladaptive substance use in which a test agent is contacted with a cell that expresses H-ras in the absence of test agent. Preferably, the method is carried out using an in vitro assay. In such assays, the test agent can be contacted with a cell in culture or present in a tissue. The level of H-ras function is determined in the presence and absence (or presence of a lower amount) of test agent to identify any test agents that alter the level. If the level of H-ras function is altered, the test agent is selected as a potential modulator of a symptom of maladaptive substance use. In a preferred embodiment, an agent that reduces H-ras function is selected as a potential inhibitor of one or more symptoms of maladaptive substance use.

Cells or tissues useful for screening based on H-ras function include any of those described above in connection with screening based on levels of H-ras or H-ras polynucleotides.

H-ras function can be measured using any suitable assay for any H-ras response or function. Examples of suitable assays include the measurement of: H-ras modification by, e.g., FPT, H-ras membrane association, H-ras guanine nucleotide exchange, and H-ras' intrinsic GTPase activity.

E. Test Agent Databases

In a preferred embodiment, generally involving the screening of a large number of test agents, the screening method includes the recordation of any test agent selected in any of the above-described prescreening or screening methods in a database of agents that may modulate a symptom of maladaptive substance use in a subject.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

F. Test Agents Identified by Screening

When a test agent is found to alter the level of Ras (e.g., H-ras) polypeptides, polynucleotides, or function, a preferred screening method of the invention further includes combining the test agent with a carrier, preferably a pharmaceutically acceptable carrier, such as are described above. Generally, the concentration of test agent is sufficient to alter the level of H-ras polypeptides, polynucleotides, or function when the composition is contacted with a cell. This concentration will vary, depending on the particular test agent and specific application for which the composition is intended. As one skilled in the art appreciates, the considerations affecting the formulation of a test agent with a carrier are generally the same as described above with respect to methods of mitigating a symptom of maladaptive substance use.

In a preferred embodiment, the test agent is administered to an animal to measure the ability of the selected test agent to modulate a symptom of maladaptive substance use in a subject, as described in greater detail below.

G. Screening for Modulation of a Symptom of Maladaptive Substance Use

The invention also provides a method of screening for an agent that that modulates a symptom of maladaptive substance use in a subject. The method entails selecting a Ras (e.g., H-ras) modulator as a test agent, and measuring the ability of the selected test agent to modulate maladaptive substance use in a subject. Any agent that modulates H-ras and that can be administered to a subject can be employed in the method. Test agents selected through any of the prescreening or screening methods of the invention can be tested for modulation of maladaptive substance use. Alternatively, known H-ras modulators can be employed. In a preferred embodiment, the selected test agent is H-ras inhibitor.

Test agents can be formulated for administration to a subject as described above for H-ras inhibitors.

The subject of the method can be any individual that has H-ras and in which symptoms of maladaptive substance use can be measured. Examples of suitable subjects include research animals, such as Drosophila melanogaster, mice, rats, guinea pigs, rabbits, cats, dogs, as well as monkeys and other primates, and humans. In preferred embodiments, an animal model established for studying particular drug-related effects or behaviors is employed. For instance, the animal model can be one that tests consumption of a substance, as described in Example 1.

The test agent is administered to the subject before, during, and/or after administration of the substance of interest, and the subject is tested or observed to determine whether the test agent modulates a particular symptom of maladaptive substance use. Test agents can be administered by any suitable route, as described above for H-ras inhibitors. Generally, the concentration of test agent is sufficient to alter the level of H-ras polypeptides, polynucleotides, or function in vivo.

The substance and symptom of maladaptive substance use studied can be any of those described above (e.g., drug reward, incentive salience for the drug, drug craving, drug seeking, and drug consumption). The substance is administered by any suitable route and in an amount sufficient to produce the symptom under examination. The symptom is measured and compared with that observed in the absence of test agent and/or in the presence of a lower amount of test agent.

Method of Assessing Risk of Maladaptive Substance Use

Another aspect of the invention is a method of assessing a subject's risk for maladaptive substance use. The method entails measuring one of several Ras- (e.g., H-ras-) related parameters in a biological sample from the subject. Suitable parameters include H-ras polypeptides, polynucleotides, and/or function. The considerations affecting sample preparation and assay are as described above, with the additional consideration that sample collection is preferably minimally invasive to the subject.

The risk for maladaptive substance use is directly correlated with each of these levels. To determine whether the subject has a normal, elevated, or reduced risk, the level measured for the selected H-ras parameter is compared to that of an appropriate matched (e.g., for age, sex, etc.) control population. The control population can be representative of the general population to allow a determination of risk of the individual subject as compared to, for example, the average risk in the general population.

If a subject is determined to have a high risk for maladaptive substance use, H-ras inhibitor can be administered to the subject to reduce this risk. Preferably, the inhibitor dose is sufficient to reduce the levels of H-ras polypeptides, polynucleotides, and/or function to within a normal range (i.e., the range observed in the control population).

Kits

The invention also provides kits useful in practicing the methods of the invention. In one embodiment, a kit of the invention includes H-ras inhibitor in a suitable container. In a variation of this embodiment, H-ras inhibitor is formulated in a pharmaceutically acceptable carrier. The kit preferably includes instructions for administering H-ras inhibitor to a subject to mitigate a symptom of maladaptive substance use.

In another embodiment, the kit is a diagnostic kit for use in assessing a subject's risk for maladaptive substance use. The kit includes at least one component that specifically binds to H-ras polypeptides or polynucleotides. This binding component can be used to detect the presence of its binding partner in a biological sample from the subject. In a preferred embodiment, the binding component is labeled with a detectable label or, alternatively, the kit includes a labeling component that is capable of binding to, and thereby labeling, the binding component when the diagnostic method of the invention is carried out. The kit preferably includes instructions for carrying out the diagnostic method of the invention.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered to illustrate, but not to limit, the claimed invention.

### Example 1

### Ethanol Alters Trafficking and Functional NMDA Receptor NR2 Subunit Ratio Via H-Ras

Abstract

The N-methyl D-Aspartate receptor (NMDAR) plays a critical role in synaptic plasticity, and is one of the main targets for alcohol (ethanol) in the brain. Trafficking of the NMDAR is emerging as a key regulatory mechanism that underlies channel activity and synaptic plasticity. This study shows that exposure of hippocampal neurons to ethanol increases the internalization of the NR2A but not NR2B subunit of the NMDAR via the endocytic pathway. In particular, ethanol exposure results in NR2A endocytosis through the activation of H-ras and the inhibition of the tyrosine kinase Src. Importantly, ethanol treatment alters functional subunit composition from NR2A/NR2B- to mainly NR2B-containing NMDARs. These results suggest that addictive drugs such as ethanol alter NMDAR trafficking and subunit composition. This may be an important mechanism by which ethanol exerts its effects on NMDARs to produce alcohol-induced aberrant plasticity.

Introduction

Since changes in the phosphorylation-state of NMDAR subunits are important for both the trafficking of NMDARs and ethanol's actions, this study examined whether the modulation of NMDAR activity by ethanol was due to changes in the trafficking of the receptor's subunits. This work demonstrates that, in the hippocampus, acute exposure to ethanol specifically increases the internalization of the NR2A subunit via ethanol-mediated activation of H-Ras. Importantly, as a consequence of the NR2A internalization, the remaining NR2B-containing NMDARs are predominant in mediating the excitatory postsynaptic currents (EPSCs) in the presence of ethanol.

Methods

Materials

The anti-NR2A, anti-NR2B, anti-actin antibodies, and all secondary antibodies, were purchased from Santa Cruz Biotechnologies (Santa Cruz, Calif.). Phosphatase inhibitor cocktail was purchased from Sigma (St. Louis, Mo.). Protease inhibitor tablets were purchased from Roche Applied Science (Mannheim, Germany). Anti-GluR1 antibodies were purchased from Chemicon (Temecula, Calif.). Raf-1-Ras binding domain GST agarose beads, H-Ras dominant-negative cDNA, anti-Src, anti-TrkB and pan anti-Ras antibodies were purchased from Upstate Biotechnologies (Lake Placid, N.Y.). Anti-[pY418]Src was purchased from Biosource (Camarillo, Calif.). The cross-linking reagent bis(sulfosuccinimidyl)suberate (BS³), and protein determination kit (bicinchoninic acid assay (BCA)) were purchased from Pierce (Rockford, Ill.). Chymotrypsin was purchased from Worthington Biochemicals (Lakewood, N.J.), and 4-(2-aminoethyl)benzenesulfonylfluoride (AEBSF) was purchased from Calbiochem (San Diego, Calif.). LipofectAMINE PLUS and DMEM were purchased from Invitrogen (Grand Island, N.Y.). Fetal Bovine Serum (FBS) was purchased from Hyclone (Logan, Utah). Dexamethasone was purchased from Sigma (St. Louis, Mo.) and ketamine was purchased from Abbott Laboratories (North Chicago, Ill.). L(−tk) cells stably transfected with NR1+NR2A or NR1+NR2B were a generous gift from Merck Sharp and Dohme and PSD-95-GFP cDNA was a gift from Dr. David Bredt (UCSF). Tat-peptide (YGRKKRRQRRR: SEQ ID NO:2) was synthesized by Syn Pep (Dublin, Calif.). The purity of the peptide was greater than 90%, and the integrity was determined by Mass spectroscopy.

Mice

C57BL/6J, Src+/− (B6.129S7-Src^{tm1Sor}) and Fyn−/− (B6.129S7-Fyn^{tm1Sor}) mice were purchased from Jackson Laboratories. Fyn−/−mice were mated in-house with C57BL/6J mice to generate Fyn+/− mice, which were subsequently mated to generate Fyn−/− and their corresponding Fyn+/+ littermates. Similarly, Src+/− littermates were mated in-house to generate Src+/− and their corresponding Src+/+ littermates. The genotypes of the mice were determined by PCR analysis of genomic DNA extracted from tail tissue. The mean age of animals used for biochemical studies was 10-12 weeks. Experimental protocols involving the use of vertebrate animals were approved by the Gallo Research Center subcommittee on Research Animal Care and met National Institutes of Health guidelines.

Preparation of Tat-H-Ras Dominant Negative(DN) Fusion Protein

pTAT-H-Ras DN was expressed in, and purified from, Escherichia coli as previously described (17).

Cell Culture

L(−tk) were cultured on 100 mm plates. When cells reached 75% confluency, they were transfected with 5 μg of PSD95-GFP cDNA using LipofectAMINE PLUS in accordance with the manufacturer's instructions. 24 hrs after transfection, the expression of the NMDAR subunits NR1+NR2A or NR1+NR2B was induced with 1 mM dexamethasone in DMEM containing 10% FBS, and 0.5 mM ketamine as previously described (21). After 3 days of induction, the cross-linking or immunoprecipitation studies were conducted. Homogenates were obtained as described in (17). The expression of NR1 and PSD-95 was routinely verified (data not shown).

Preparation of Hippocampal Slice Homogenates

Transverse hippocampal slices (250 mm) were prepared from 10- to 12-week-old C57BL/6J mice. Slices were allowed to recover for 90 min in artificial cerebrospinal fluid (aCSF) saturated with 95% O₂/5% CO₂ containing (in mM): 126 NaCl, 1.2 KCl, 1.2 NaH₂PO₄, 1.2 MgCl₂, 2.4 CaCl₂, 18 NaHCO₃, and 11 glucose. Following recovery, slices were treated with aCSF (control) or ethanol at room temperature, after which the slices were rinsed in cold aCSF, and used either for cross-linking experiments, for Ras activity assay, or for immunoprecipitation. In experiments using Tat-H-Ras DN or the Tat-peptide, the slices were pretreated with either aCSF (control) or 2 μM Tat-H-Ras DN or Tat-peptide for 2 hrs at room temperature, followed by ethanol treatments.

Cross-Linking Studies

Cross-linking was performed as described previously (22), with minor modifications. Following treatment, the slices were placed in cold aCSF in the presence or absence of 2 mg/ml of the cross-linking reagent BS³ for 45 min at 4° C. while shaking. Slices were then rinsed three times with cold aCSF containing 20 mM Tris (pH 7.6), followed by sonication in cold homogenization buffer (10% SDS, 10 mM EDTA, 100 mM Tris (pH 8.0), and protease and phosphatase inhibitor cocktails). The protein concentration was determined using the BCA assay, followed by SDS-PAGE and Western blot analysis. Data are expressed as ratio of normalized surface expressed or internalized receptors to the total receptor levels.

Chymotrypsin Experiments

Chymotrypsin experiments were performed as described previously (22), with minor modifications. Following recovery, slices were treated with aCSF or 0.5 mg/ml chymotrypsin at 32° C. for 5 min. The chymotrypsin reaction was stopped by three washing steps in aCSF containing 2 mM AEBSF (a chymotrypsin inhibitor), followed by sonication in cold homogenization buffer (10% SDS, 10 mM EDTA, 100 mM Tris (pH 8.0), and protease and phosphatase inhibitor cocktails). The protein concentration was determined using the BCA assay, followed by SDS-PAGE and Western blot analysis.

Ras Activity Assay

The treated hippocampal homogenates (250 μg) were incubated with 15 ml of Raf-1-Ras binding domain GST-agarose for 45 min at 4° C. while rotating, as per manufacturer's instructions. The beads were then washed three times with lysis/wash buffer (provided by manufacturer), resuspended in 2× sample buffer, resolved by SDS-PAGE and analyzed by Western blot with pan anti-Ras antibodies.

Immunoprecipitation

The treated slices were homogenized in ice-cold buffer containing 1% deoxycholate, protease and phosphatase inhibitors and (in mM): 250 sucrose, 20 Tris-HCl (pH 7.5), 2 EDTA and 10 EGTA. 500 μg of the protein was diluted with 1× immunoprecipitation buffer (1% Triton X-100 and (in mM): 150 NaCl, 10 Tris HCl (pH 7.4), 1 EDTA, 1 EGTA and 0.2 sodium orthovanadate) and incubated with 5 μg of the appropriate antibody, followed by an overnight incubation at 4° C. with protein G agarose beads. The beads were extensively washed, re-suspended in 2× sample buffer and boiled for 5-10 min. The samples were then subjected to SDS-PAGE followed by Western blotting with the appropriate antibodies.

Western Blot Analysis

Proteins were separated by SDS-PAGE and blotted onto a nitrocellulose membrane. After blocking, the membranes were incubated with the specific primary antibody, followed by horseradish peroxidase-conjugated secondary antibody. Detection was obtained followed by enhanced chemiluminescent reaction (Amersham Biosciences), and processed using the STORM PhosphoImager (Amersham Biosciences). Results were quantified by NIH Image 1.61.

Electrophysiology

Electrophysiological recordings were performed in C57BL/6J mice ranging from P21 to P26. Briefly, mice were anesthetized with halothane and decapitated as per University of California, San Francisco animal care guidelines. Horizontal sections of the hippocampus (230-300 mm) were prepared with a vibratome (Leica, Nussloch, Germany). Slices were placed in a holding chamber and allowed to recover for at least 1 hr before being placed in the recording chamber and superfused with aCSF saturated with 95% O₂/5% CO₂ and containing (in mM): 119 NaCl, 1.6 KCl, 1.0 NaH₂PO₄, 1.3 MgCl₂ (for EPSCs) or 0.01 MgCl₂ (for field excitatory postsynaptic potentials (fEPSPs)), 2.5 CaCl₂, 26.2 NaHCO₃ and 11 glucose. Picrotoxin (100 μM) was added to block gamma-aminobutyric acid-A (GABA_{A}) receptor-mediated inhibitory synaptic transmission, and CNQX (for EPSCs) or NBQX (for fEPSPs) (10 μM) was added to block α-amino-3-hydroxy-5-methyl-4-isoxalepropionate (AMPA)/Kainate receptors. Cells were visualized using infrared differential interference contrast video microscopy.

Whole-cell voltage clamp-recordings were made using an Axopatch ID or a MultiClamp 700A amplifier (Axon Instruments, Union City, Calif.). Electrodes (3.5-5.0 MΩ) contained (in mM): 120 Cesium methansulfonate, 20 HEPES, 0.4 EGTA, 2.8 NaCl, 5 TEA-Cl, 2.5 MgATP, and 0.25 NaGTP (pH 7.2-7.3), buffer at 270-285 mOsm. Series resistance (10-30 MΩ) and input resistance were monitored on-line with a 4 mV depolarizing step (50 ms) given just after every afferent stimulus. Recordings from the CA1 region of the hippocampus were obtained with a bipolar stimulating electrode that was placed 100-300 μm rostral to the recording electrode and was used to stimulate excitatory afferents at 0.1 Hz. Neurons were voltage-clamped at +40 mV to evoke NMDAR-mediated EPSCs. At this potential, NMDARs have a maximal open probability and the Mg²⁺ block is removed. EPSCs were filtered at 2 kHz, digitized at 5-10 kHz and collected on-line using Igor Pro software (Wavemetrics, Lake Oswego, Oreg.) or pClamp 9 (Axon Instruments, Union City, Calif.). NMDAR example traces were constructed by averaging 20 EPSCs (200 sec) elicited at +40 mV during baseline, ethanol application, drug application or drug application with ethanol. To calculate the decay time (τ), Igor Pro software was used to fit exponential decay curves to averaged NMDAR-mediated EPSCs using the formula: y=A₁exp(−invTau₁*x)+A₂exp(−invTau₂X).

fEPSPs were recorded using a MultiClamp 700A amplifier (Axon Instruments, Union City, Calif.) with whole-cell patch pipettes filled with 1 M NaCl and 25 mM HEPES. To evoke fEPSPs, Schaffer collateral/commissural afferents in hippocampal CA1 region were stimulated with 0.1 Hz pulses using steel bipolar microelectrodes at intensities adjusted to produce an evoked response that was 50% of the maximal recorded fEPSP for each recording. fEPSPs were filtered at 1 kHz, digitized at 10 kHz and collected on-line using with pClamp 9 (Axon Instrument, Union City, Calif.). NMDAR traces were constructed by averaging 6 fEPSPs (1 min) during the whole recordings.

Statistical Analysis

For biochemistry experiments, results were obtained from at least 3 different animals as indicated in the figure legends. For electrophysiology experiments, n values represent the number of individual slices from at least 3 individual animals as indicated in the figure legends. Data are expressed as the mean±S.D. (biochemistry) or S.E.M (electrophysiology) and were analyzed using a Student's t-test or one-way ANOVA followed by a Newman-Keuls post-hoc test.

Results

Acute Ethanol Exposure Results in NR2A Subunit Internalization

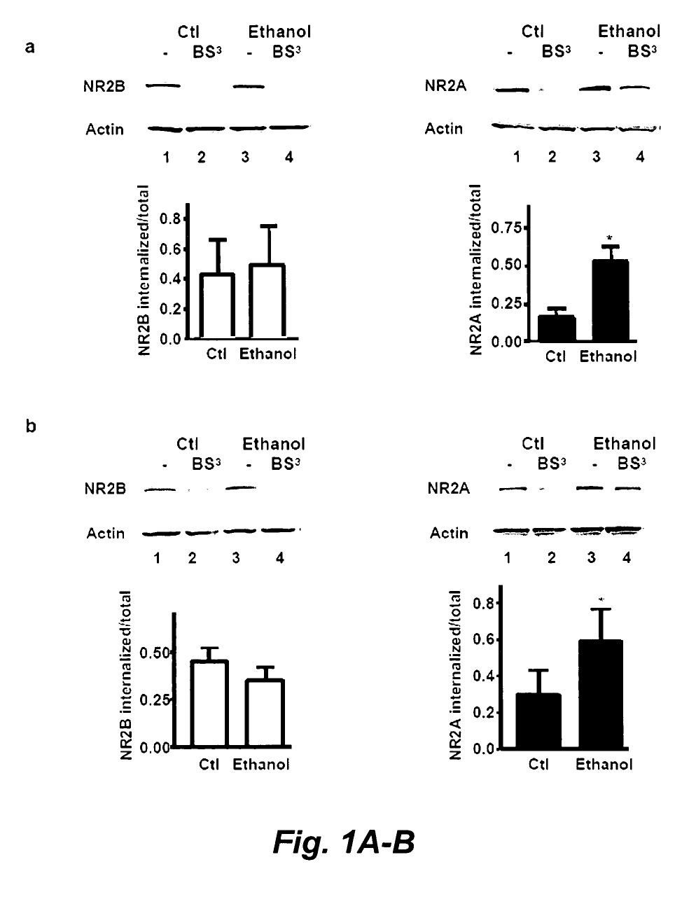

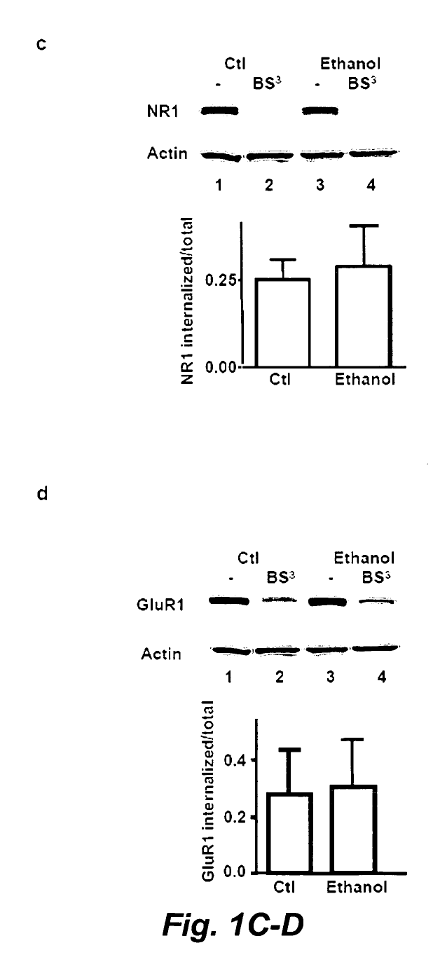

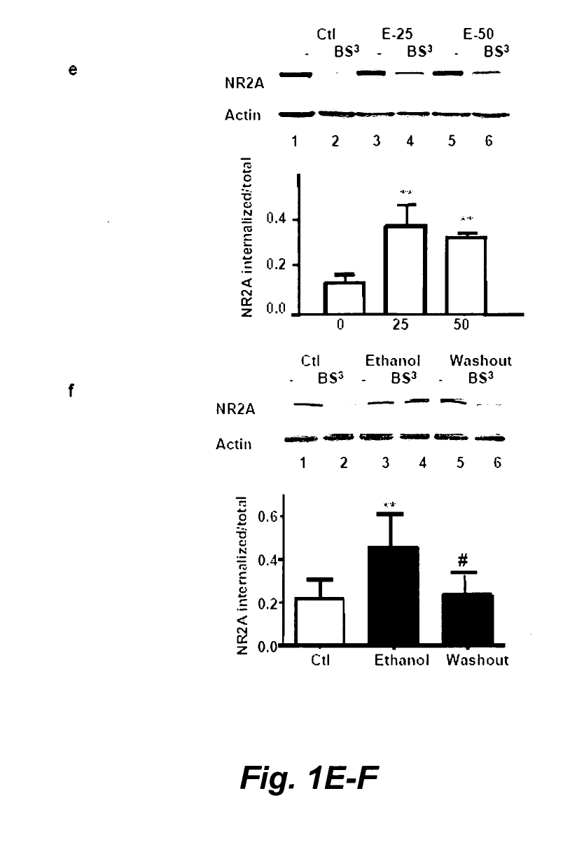

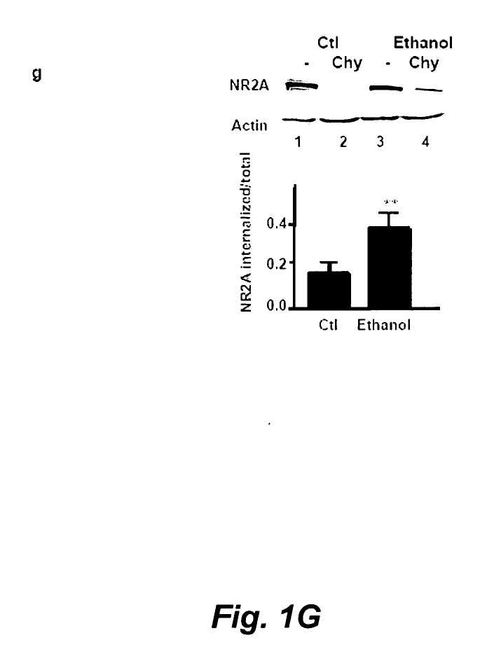

To investigate whether acute ethanol treatment alters the surface expression of the NMDAR subunits, the membrane-impermeable cross-linking agent BS³ was utilized. The cross-linker produces high molecular weight aggregates of the surface receptors that cannot enter the SDS-PAGE gel, and therefore the intracellular pool of receptors can be quantified (22). Incubation of hippocampal slices with ethanol (100 mM) did not alter the membranal localization of the NR2B subunit (FIG. 1a, left, lanes 2 vs. 4), but led to a significant increase in the intracellular level of NR2A, which was accompanied by a corresponding decrease in surface-expression of the subunit (FIG. 1a, right, lanes 2 vs. 4). These changes were not due to alteration in the expression level of NR2A (FIG. 1b, lanes 1 vs. 3). To confirm that the ethanol-mediated effect on the subcellular localization of the NMDARs is specific for the NR2A subunit, the cross-linking experiments were repeated in mouse L(tk−) cell lines that stably express either the NR2A+NR1 or NR2B+NR1 under the control of a dexamethasone-inducible promoter (21). To ensure that the NMDARs are properly compartmentalized, the cells were also transiently transfected with the scaffolding protein postsynaptic density protein 95 (PSD-95). As shown in FIG. 1b, ethanol exposure resulted in an increase in the internalized pool of the NR2A (FIG. 1b, right, lanes 2 vs. 4), but not NR2B (FIG. 1b, left, lanes 2 vs. 4) subunits. In addition, no change was observed in the surface expression of the NR1 subunit of the NMDAR, or the GluR1 subunit of the AMPAR (FIGS. 1c and d, lanes 2 vs. 4). These results suggest that ethanol's actions are specific for the NR2A subunit. Next, it was determined whether the ethanol-mediated NR2A internalization could still be detected at lower concentrations of ethanol, and whether the effect can be reversed upon ethanol washout. Ethanol concentrations as low as 25 mM induced the reduction in the surface expression of the subunit, and a corresponding increase in the immunoreactivity of the subunit in the intracellular compartment (FIG. 1e, lanes 2 vs. 4 and 6). This change in the subcellular localization of NR2A was not observed after ethanol was treated for 15 min, and then washed out from the slice preparation, and the slices were allowed to recover for an additional 15 min. (FIG. 1f, lanes 4 vs. 6). Finally, the results were confirmed using another independent method. Following aCSF or ethanol treatment, the slices were treated with the serine protease, chymotrypsin, which is excluded from the interior of the cell due to its large size. Therefore, chymotrypsin cleaves the surface receptors, and only the intracellular pool of receptors is then detected by Western blot analysis (22). This method has previously been used to show LTP-mediated changes in surface expression of the NMDAR subunits (10). FIG. 1g (lanes 2 vs. 4) demonstrates that ethanol exposure significantly increased the internalization of the NR2A subunits, however, ethanol had no effects on the surface or intracellular levels of NR2B (data not shown). Taken together, these results show that in hippocampal slice preparation ethanol treatment results in the specific internalization of the NR2A subunit of the NMDARs.

Ethanol Exposure Increases the Association Between the NR2A Subunit and the AP2 Adaptor Protein Complex

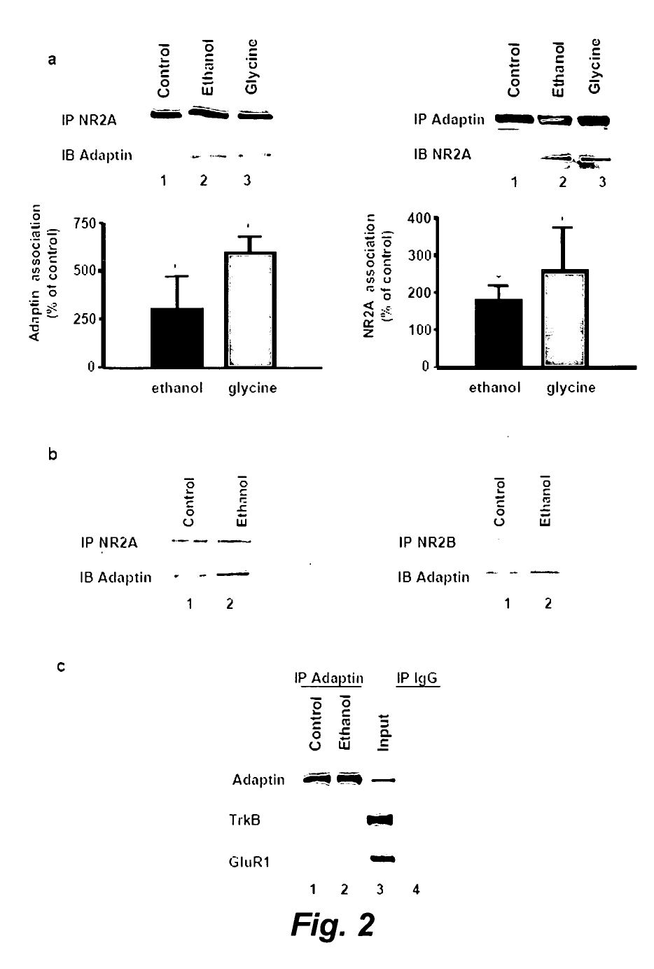

The intracellular tails of the NR2 subunits contain internalization motifs that mediate clathrin-dependent receptor endocytosis (23). To determine if ethanol exposure potentiates the endocytic pathway to internalize NR2A subunits, the question of whether, in the presence of ethanol, the subunit interacts with adaptin β2 (a component of the adaptor protein complex 2 (AP-2) involved in clathrin-dependent receptor endocytosis) was examined. Similar to glycine, which was recently shown to prime the receptor for internalization (12) (FIG. 2a, lane 3), exposure to ethanol (100 mM, 15 min) significantly increased the association of adaptin β2 with NR2A and vice-versa (FIG. 2a, lanes 1 vs. 2). To address the question of specificity of adaptin interaction with the NR2A, similar studies were conducted in the mouse L(−tk) cells that express either the NR1+NR2A (FIG. 2b, left) or NR1+NR2B (FIG. 2b, right) subunits together with PSD-95. Ethanol treatment resulted in an increase in the association of NR2A with adaptin β2 (FIG. 2b, left, bottom panel), but no change in NR2B-adaptin association was observed (FIG. 2b, right, top panel). Finally, to determine if the association with adaptin β2 is specific for an NMDAR subunit, the question of whether ethanol alters the association of adaptin β2 with the AMPAR subunit GluR1, and the brain derived neurotrophic factor receptor subunit TrkB was tested. There was no change in the interaction between these proteins and adaptin β2 (FIG. 2c, middle and low panels). These results suggest that ethanol treatment caused the recruitment of the endocytic machinery to the NMDAR, leading to NR2A endocytosis.

Ethanol-Mediated Increase in Ras Activity is Necessary for NR2A Endocytosis

Next, the mechanism for ethanol-mediated NR2A internalization was determined. Tyrosine dephosphorylation of NR2A subunits can lead to the internalization and rundown of NMDAR activity (16), and the surface expression of the NR2A but not the NR2B subunit is negatively regulated via the association of the small GTP binding protein H-Ras with Src kinase, and consequent inhibition of Src activity (17). Both Src and H-Ras are important regulators of NMDAR function and synaptic plasticity (24-26). It was therefore hypothesized that in the presence of ethanol, NR2A is internalized as a result of H-Ras activation and the subsequent inhibition of Src activity that prevents the tyrosine phosphorylation of the subunit.

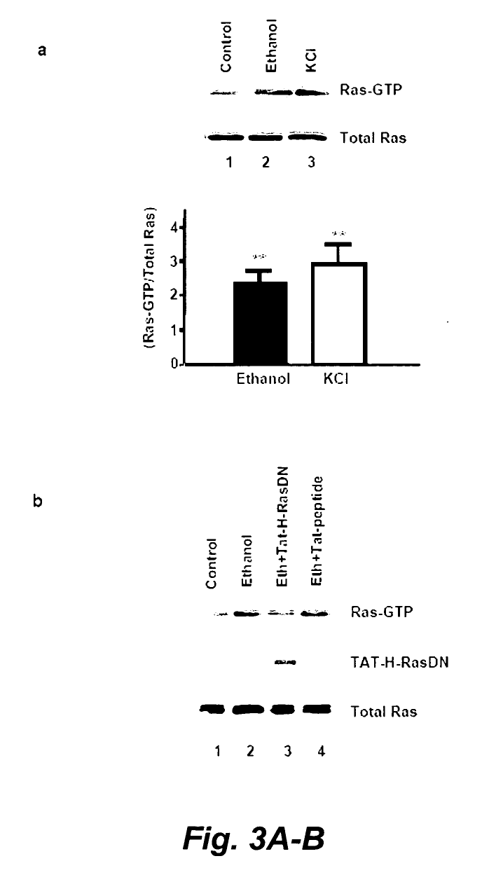

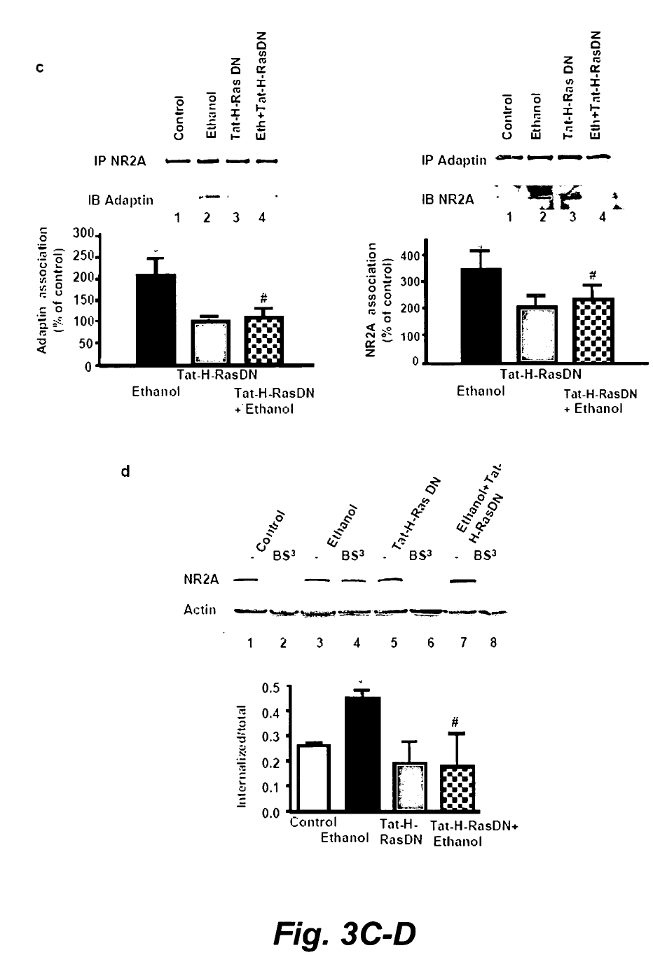

To measure H-Ras activity, the level of GTP-bound active Ras association with the Raf-1-Ras binding domain was determined, in the presence or absence of ethanol. Similar to KCl treatment (FIG. 3a, lanes 1vs. 3), which increases Ras activity, ethanol exposure (100 mM, 15 min) increased Ras-GTP levels by at least 2-fold over the control (FIG. 3a, lanes 1 vs. 2), suggesting that ethanol treatment elevates total Ras activity. The Ras activity assay does not differentiate between the Ras family members (H-Ras, K-Ras and N-Ras). However, since in the adult mouse brain, H-Ras expression is two to five times higher than the other isoforms (27), it was likely that at least part of the Ras activity detected in the presence of ethanol is that of H-Ras. If H-Ras activity is important for NR2A internalization, then inhibiting H-Ras should prevent ethanol's actions on NR2A endocytosis. To inhibit H-ras activity, the dominant negative form of H-Ras (H-Ras DN) was transduced into hippocampal slices using the Tat-fusion protein transduction system (28). The H-Ras DN sequesters the guanine nucleotide exchange factors necessary for Ras activation and thereby inhibits the activation of endogenous Ras (29). First, it was determined whether Tat-H-Ras DN inhibits the ethanol-mediated increase in endogenous Ras activity. As shown in FIG. 3b (lanes 2 vs. 3), pre-incubation of hippocampal slices with 2 mM Tat-H-Ras DN decreased endogenous Ras activity, but the Tat-peptide (2 μM), which was used as a control, did not (FIG. 3b, lanes 2 vs. 4). Next, the association of the NR2A subunits to adaptin β2 was measured in the absence or presence of Tat-H-Ras DN. Tat-H-Ras DN alone had no effect on the interaction between NR2A and adaptin β2 or vice-versa (FIG. 3c, lane 3); however, Tat-H-Ras DN blocked the ethanol-mediated association of adaptin β2 with NR2A or vice-versa (FIG. 3c, lanes 2 vs. 4), and ethanol-mediated internalization of the NR2A subunits (FIG. 3d, lanes 4 vs. 8). Taken together, these results suggest that the ethanol-mediated activation of H-Ras is required for NR2A internalization.

Src Inhibition is Necessary for the Internalization of NR2A

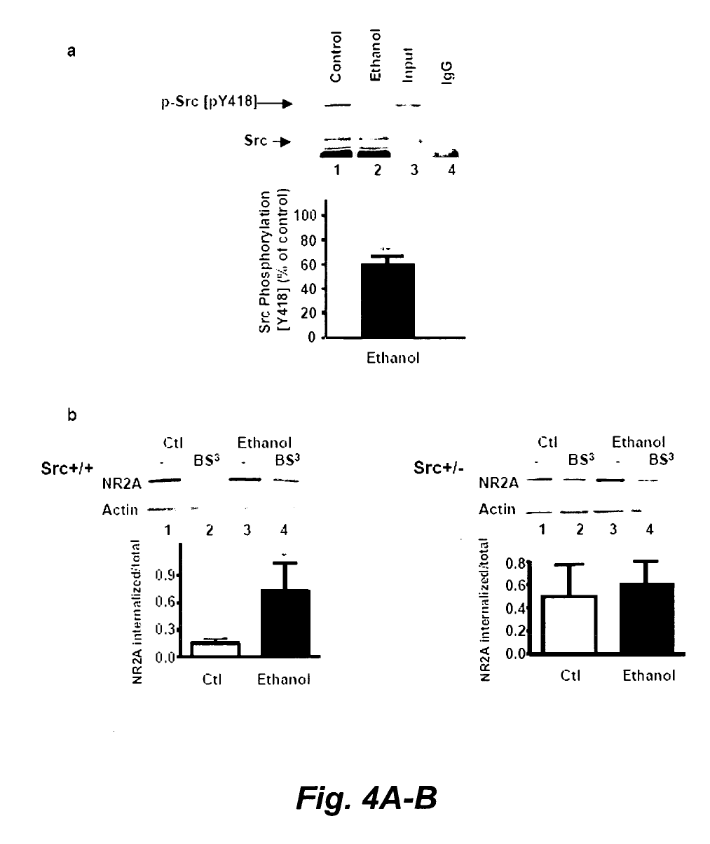

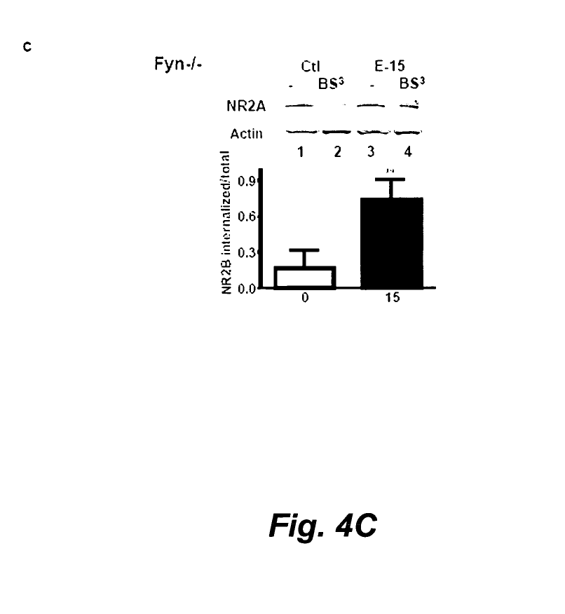

Active H-ras interacts with Src to inhibit the activity of the kinase, resulting in the reduction of phosphorylation and surface expression of NR2A (17). To test whether Src is inhibited in the presence of ethanol, the activity of the kinase was measured in the absence or presence of ethanol by immunoprecipitating Src, and subsequently detected the level of the enzyme with anti-[pY418]Src antibody that recognizes only the kinase in its active state. Following ethanol exposure, there was an approximately 40% decrease in the levels of active phosphorylated Src (FIG. 4a, lanes 1 vs. 2), suggesting that, in the presence of ethanol, Src kinase activity is indeed inhibited. Next, to determine whether the ethanol-mediated NR2A internalization was dependent upon the inhibition of Src, the surface expression of NR2A was tested in the presence or absence of ethanol in hippocampal slices from Src heterozygote mice (Src+/−), which express 50% of the Src gene, and their corresponding littermate controls (Src+/+). Similar to the results obtained from C51BL/6J mice (FIG. 1b), ethanol significantly increased the internalization of the NR2A subunits in Src+/+ mice (FIG. 4b, left, lanes 2 vs. 4). In contrast, NR2A was internalized under basal conditions in Src+/− mice, and no additional changes were observed upon exposure to ethanol (FIG. 4b, right, lanes 2 vs. 4), suggesting that the reduction of Src expression occludes ethanol-mediated internalization of NR2A. To determine whether ethanol-induced internalization is mediated specifically via the inhibition of Src, the experiment was repeated in Fyn−/− and Fyn+/+ mice. Ethanol exposure resulted in a significantly enhanced NR2A internalization in both the Fyn+/+ and Fyn−/− mice (data not shown, and FIG. 4c, lanes 2 vs. 4). These results suggest that the inhibition of Src, but not of Fyn, activity is necessary for the internalization of the NR2A subunit in the presence of ethanol.

Characterization of Ethanol's Actions on NMDAR-Mediated Activity in the CA1 Region of the Hippocampus

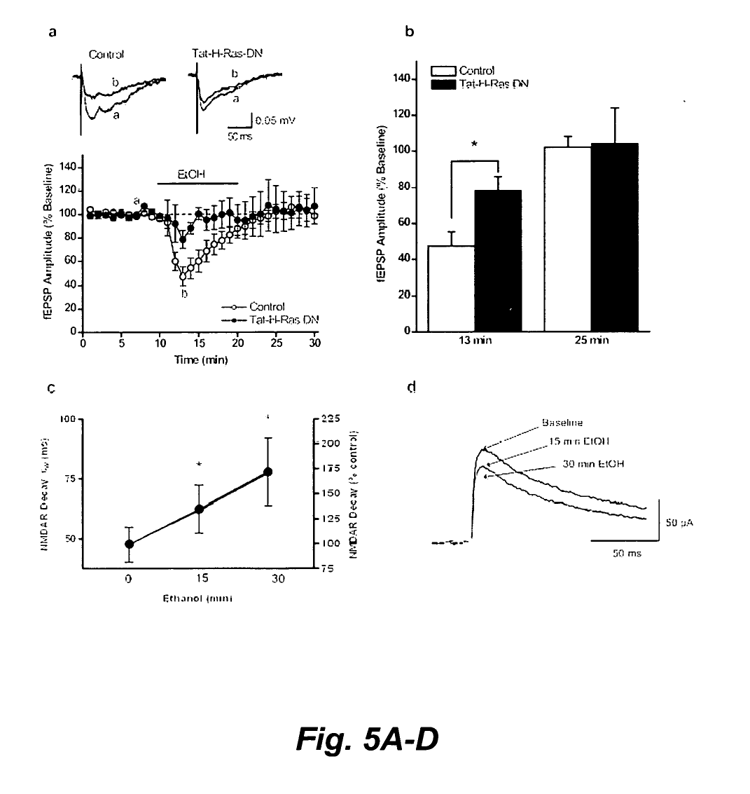

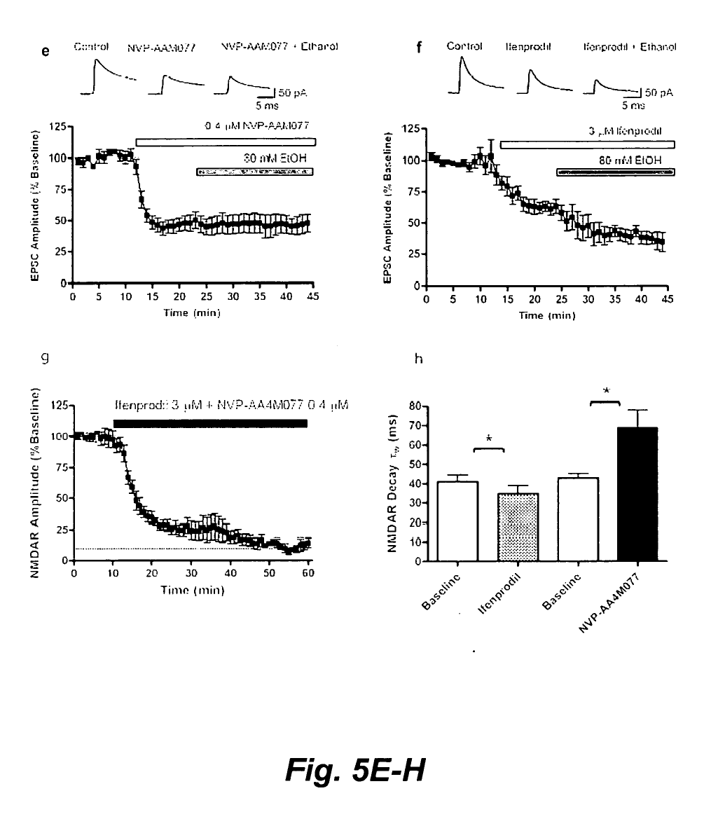

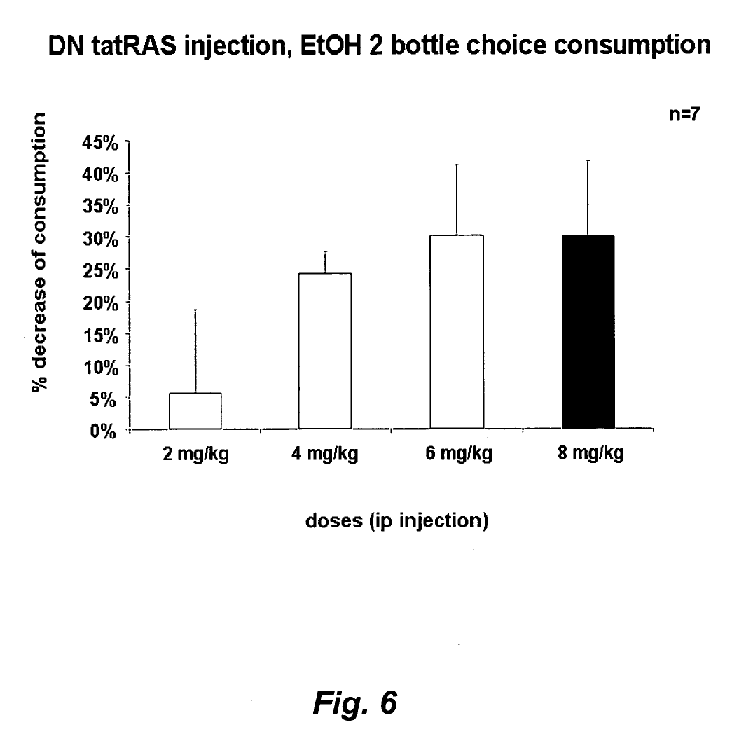

It is well established that ethanol exposure inhibits NMDAR-mediated activity in neuronal preparations (30-32). Similarly, bath application of 80 mM ethanol inhibited NMDAR-mediated fEPSPs (47+8%, n=11, FIG. 5a, b, white circles, white bar) and EPSCs (37±7%, n=9, data not shown) in the hippocampus. In line with these previous studies (30-32) and others, incubation with low ethanol concentration (25 mM) inhibited ethanol-mediated EPSCs by 24±6% (n=18) (data not shown), and as has previously been reported (18,20,33-35), there was an acute desensitization to the inhibitory effects of ethanol during field-recording of channel activity (FIG. 5a).

Inhibition of H-Ras Reduced the Inhibitory Actions of Ethanol on NMDAR-Mediated fEPSP

To test whether ethanol-induced internalization of the NR2A subunit via the activation of H-Ras contributes to the inhibitory actions of ethanol on the NMDAR in the hippocampus, NMDAR-mediated fEPSPs were measured in the presence and absence of ethanol, and in the presence and absence of Tat-H-Ras DN. Ethanol-mediated inhibition of fEPSPs was reduced by pre-incubation of the slices with 1 μM Tat-H-Ras DN (22+8%, n=8, *p<0.05, FIG. 5a, b, black circles, black bars), suggesting that H-Ras mediated internalization of the NR2A subunit contributes to the inhibitory effects of ethanol on NMDAR function.

Ethanol Exposure Alters the Decay Time of the NMDARs

Since ethanol exposure decreased the surface expression of the NR2A but not NR2B subunits, it was hypothesized that the internalization of NR2A in the presence of ethanol would reduce the contribution of this subunit to the activity of the NMDARs. Alteration in NMDAR subunit composition is reflected by a specific change in the decay time of evoked NMDAR-mediated EPSCs; in particular, the NR2A-containing NMDAR exhibits a faster decay time relative to the NR2B-containing NMDAR (2,36). Therefore, the question of whether ethanol would slow the decay of NMDAR EPSCs was examined. As predicted, the average values of the fast and slow components of the decay-time constant (τ_{f} and τₛ, respectively) of the NMDAR EPSCs were slower after 15 or 30 min application of 80 mM ethanol (Table 1, FIG. 5c,d). Ethanol slowed the weighted decay time by 33±14% (τ_{w}=62±10 ms, n=6) after 15 min and 70±29% (τ_{w}=78±14 ms; n=6; *p<0.05) after 30 min compared to the control (τ_{w}=47±7 ms; n=6; *p<0.05) (FIG. 5c,d), suggesting that the contribution of the NR2A-containing NMDARs to the activity of the channel is reduced.

TABLE 1
Decay constants of NMDAR EPSCs after 0, 15, 30 min. exposure
to ethanol (80 mM) fit with double exponential curve.

| Treatment | | τ_{F} (ms) | τ_{S} (ms) | % Fast | τ_{W} (ms) |
|---|---|---|---|---|---|
| 0 | min | 30.3 ± 2.0 | 158.4 ± 25.3 | 13.5 ± 3.0 | 47.4 ± 7.4 |
| 15 | min | 34.5 ± 2.1 | 256.3 ± 51.8* | 12.5 ± 3.1 | 62.2 ± 10* |
| 30 | min | 42.4 ± 4.4* | 354.0 ± 91.1 | 112.6 ± 2.0 | 77.8 ± 14.1* |

All values expressed as means±S.E.M. * significantly different than 0 min, same developmental age, P<0.05, paired t-test. n=6.

Ethanol Treatment Alters NMDAR Subunit Composition of Functional Channels

If the inhibition of channel activity is due to ethanol-induced internalization of the NR2A subunits, then the majority of the remaining NMDARs should consist of the NR2B-containing receptors. To test this possibility, the NR2A antagonist, NVP-AAM077, was applied at concentrations that have previously been shown to distinguish between the hippocampal NR2A- and NR2B-containing NMDARs (37), and determined the degree of ethanol-mediated inhibition of the remaining current. NVP-AAM077 (0.4 μM) applied to slices rapidly and significantly decreased NMDAR EPSCs by 55±4% (n=6, p<0.05, FIG. 5e); however, subsequent bath application of 80 mM ethanol did not alter the remaining portion of the NMDAR EPSCs (FIG. 5e). Conversely, the inhibitory effect of ethanol on the NMDAR was measured after incubation with the selective NR2B-containing NMDA receptor antagonist ifenprodil (37). As shown in FIG. 5f, ifenprodil decreased NMDAR EPSCs by 37+5% (n=5, p<0.05), and bath application of ethanol in the presence of ifenprodil (3 μM) caused a further 36% decrease in remaining current (total reduction of 73±7%, n=6, p<0.05, FIG. 5f). Finally, ifenprodil application resulted in a further inhibition of the NMDAR-mediated EPSCs in slices treated with both ethanol and NVP-AAM077, whereas NVP-AAM077 had no effect in slices that had been first treated with ethanol and ifenprodil (data not shown). To determine whether the NMDAR subunit composition in CA1 hippocampal neurons is indeed mainly NR2A and NR2B, ifenprodil and NVP-AAM077 were co-applied. After 45 min. of application, 90% of the NMDAR mediated activity was inhibited (FIG. 5g, n=5). The stable current remaining comprised of 10.5±3.2% of the total current, and could be comprised of either/or of NR2C/D, NR3, or NR2A/NR2B hetero-trimers. The weighted decay time, fit by a double exponential curve, in the presence of ifenprodil (3 μM) significantly increased from 41.2±3.4 ms to 34.7±4.5 ms (FIG. 5h, n=7, p<0.05 paired t-test). Conversely, the weighted decay time in the presence NVP-AM077 (0.4 μM) significantly slowed from 43.0±2.1 ms to 68.7±9.4 ms (FIG. 5h, n=6, p<0.05 paired t-test). Taken together, these results suggest that the inhibition of NR2A-containing NMDAR occludes the inhibitory effect of ethanol on the activity of the channel, and that the remaining active channels after ethanol exposure are mainly NR2B-containing receptors. Thus, ethanol selectively reduces NR2A-containing NMDAR EPSCs, thereby shifting the NR2A/NR2B subunit ratio of the remaining functional receptors to mainly NR2B-containing NMDARs.

Discussion

The forward and inward trafficking of the NMDAR subunits has emerged as playing a central role in the regulation of NMDAR activity (7,8). The present study indicates that the trafficking of NMDARs is altered by ethanol. Acute exposure of hippocampal neurons to ethanol leads to the specific internalization of NR2A subunits via the endocytic pathway. The data also suggest that ethanol-induced internalization of the NR2A subunits is mediated via the activation of H-Ras and the inhibition of Src kinase activity. Importantly, the results imply that, as a consequence of the internalization, the contribution of the NR2A subunits to the activity of the NMDARs is markedly reduced.

Ethanol Treatment Results in NMDAR Subunit-Specific Internalization

Ethanol exposure results in an increase in the internalized pool of NR2A. Since ethanol exposure also resulted in an increase in the association between NR2A and adaptin β2, it is highly likely that this internalization is mediated via the endocytic pathway. However, the possibility that the increase in the internalized level of NR2A is also due to the inhibition of forward movement of the subunit in the presence of ethanol cannot be excluded. Interestingly, the localization of the NR2B subunit in the presence of ethanol was unchanged. These findings correlate with previous studies showing different modes of regulating subunit trafficking. For example, the endocytosis of NR2A and NR2B depends on different internalization motifs within the cytoplasmic tails of the subunits (23), and the NR2A and NR2B subunits differ in their rate of internalization and in their binding affinities to different μ subunits of the AP-2 adaptor protein complex (23).

There were no overall changes in the trafficking of the NR1 subunits, even though the NR2A and NR2B subunits exist as heteromeric complexes with the NR1 subunits (2). However, it is important to note that the NR1 subunits are comprised of a number of splice variants (38). The NR1 slice variants determine the localization of the subunit (39), and control the trafficking of the subunit (11,14). The CO region within the NR1 subunit was identified as an important region for regulating NMDAR sensitivity to ethanol (40,41): thus, it is possible that acute ethanol treatment may be involved in the trafficking of certain NR1 splice variants, but the overall NR1 level is unchanged. Support for this possibility comes from a study which found that prenatal exposure of cerebral cortical neurons to ethanol did not alter the total cell surface protein levels of NR1, however, the levels of the C2′ variant was markedly reduced (42).

Ras is Activated in the Presence of Ethanol

The activity of the small GTP binding protein, Ras, is increased in the presence of ethanol. H-Ras acts as a molecular “switch off” mechanism for Src-mediated NR2A (but not NR2B) phosphorylation and membrane retention (17). In the current study, ethanol increases Ras activity, and treatment with the dominant negative H-Ras mutant (Tat-H-Ras DN) blocked the ethanol-mediated increase in Ras activity and NR2A internalization and endocytosis. In addition, inhibition of H-Ras activity reverses the ethanol-mediated inhibition of NMDARsmediated fEPSPs, suggesting that increased Ras activity is necessary for ethanol-mediated NR2A internalization. Interestingly, microarray analysis of brain expression has shown that H-ras expression levels are elevated in an alcohol-preferring mouse line. Mulligan et al. (2006) Proc. Natl. Acad. Sci. USA 6368-6373

The mechanism by which ethanol exposure activates Ras is currently unknown; however, Ras activity can be stimulated by various signaling pathways, such as the activation of cAMP/PKA cascade (43,44). Since ethanol mediates its actions in part via the cAMP/PKA pathway (45), it is possible that PKA signaling is mediating Ras activation in the presence of ethanol. In addition, Ras activity can be increased via the activation of receptor tyrosine kinases (46), and acute exposure of neurons to ethanol increases the expression level and function of the brain-derived neurotrophic factor (BDNF) (47), suggesting that activation of the BDNF signaling pathway may lead to the activation of Ras.

Ethanol Exposure Prevents Src Activation

Previous studies have suggested that in order for the NR2 subunits to be internalized, the protein needs to be in a dephosphorylated state (16). The current results imply that the specific inhibition of Src activity in the presence of ethanol contributes to the internalization of NR2A subunits, presumably by preventing Src from phosphorylating the subunits. Previously, it was shown that the association between H-Ras and Src leads to the inhibition of Src activity (17); therefore, the inhibition of Src activity is likely to be mediated through the activation of H-Ras.

Both Src and Fyn kinases phosphorylate the NR2 subunits, thereby modulating the activity of the channel (26). A previous study (20) and the present results suggest opposing actions of ethanol on the activity of these two closely related kinases, leading to differential regulation of the NR2A subunit via the inhibition of Src, and the NR2B subunit via the activation of Fyn. The possible selective regulation of NR2B-containing NMDARs by Fyn, and NR2A-containing receptors by Src, is not entirely surprising since Src and Fyn are activated by different signaling pathways and have different modes of compartmentalization within the NMDAR complex (26).

Other Possible Mechanisms for the Internalization of the NR2A Subunit in the Presence of Ethanol

Previously, ethanol has been shown to mediate its effects by increasing protein phosphatase activity, thereby dephosphorylating NR2A and NR2B subunits and decreasing NMDAR activity (19). This study raises the possibility that the dephosphorylation of NR2A subunits in the presence of ethanol may also contribute to the endocytosis of the NMDAR subunits. In addition, NMDARs are localized at the synapse by protein-protein interactions with the scaffolding protein PSD-95 (48), and this interaction is thought to prevent subunit internalization via masking the AP2 binding site (49). Ethanol has been shown to alter the compartmentalization of scaffolding proteins (50), and recently anesthetics that resemble ethanol in their pharmacological properties, were found to disrupt the interaction of PSD-95 with NMDARs (51). Therefore, it is possible that ethanol exposure disrupts the specific interaction between the NR2A subunits and PSD-95, thereby allowing the interaction of NR2A with AP2.

The NR2A Subunit Contributes to the Inhibitory Actions of Ethanol on the NMDAR

Previous studies have clearly demonstrated that ethanol exposure inhibits the activity of the NMDARs (reviews; (50,52,53)). In hippocampal slices, a portion of the NR2A-containing subunits is internalized after ethanol treatment. It was therefore hypothesized that the reduction of membranal expression of the NR2A subunit may contribute to the inhibitory actions of ethanol on the activity of the channel. The main regulatory NR2 subunits in the hippocampus are the NR2A and NR2B subunits (54), which have different pharmacological properties such that NR2A-containing NMDARs have a faster decay time compared to the NR2B-containing receptors (2). The NMDAR EPSCs exhibit a slower decay time in the presence of ethanol, suggesting that the contribution of the NR2A subunit to the remaining activity of the channel is decreased. This possibility is further supported by the experiments in which application of the NR2A inhibitor, NVPAAM077, occluded the ethanol-induced inhibition of NMDAR-mediated EPSCs, whereas the NR2B-specific inhibitor, ifenprodil, did not.

The results suggesting that the NR2A subunit contributes to the inhibitory actions of the NMDARs by ethanol, are different from previous studies which suggested greater contribution of the NR2B subunit, or equal contribution of both. However, most of these experiments were done in heterologous systems such as HEK293 cells (55-60), or in Xenopus oocytes (61-64). Although these studies provide important information of the actions of ethanol on channels containing individual subunits, it is difficult to directly relate these findings to those described herein since these studies were conducted in non-neuronal systems that do not contain proteins such as PSD-95 that are required for the localization and proper function of the NMDARs. Woodward, and colleagues (58) used three expression systems—L(tk−) cells, HEK 293 cells, and Xenopus oocytes) and two methods—electrophysiological recording and Ca²⁺ imaging to evaluate the inhibition of ethanol on NR1/NR2B and NR1/NR2A. They found NR1/NR2A and NR1/NR2B have different sensitivity to ethanol in HEK293 and L(tk−) cells but not in oocytes, suggesting that assay system used influenced the degree of ethanol inhibition of recombinant NMDA receptors. Although some experiments were also done in cultured cortical (59), striatal (60), or cerebellar (65) neurons, these experiments differ from those described herein, which were conducted the experiments in hippocampal slices, and therefore the type of neurons and their developmental stage are different. Furthermore, experiments using slice electrophysiology to determine the effects of the specific NR2A and NR2B inhibitors on NMDAR sensitivity to ethanol have not been conducted before. Finally, although the experiments described herein suggest an important contribution of the NR2A subunit, they do not negate the studies showing the contribution of the NMDAR subunits. For example, studies in non-neuronal systems identified the C0 region within the NR1 subunit as an important region for regulating NMDAR sensitivity to ethanol (40,41). It is therefore possible that NMDARs that contain both NR2A or NR2B and the NR1-C0 region are highly sensitive to ethanol. It is also possible that the NR1 subunit splice variants in hippocampus may be different from those in cortical, striatal, or cerebelluar neurons, and play a role in the ethanol sensitivity of NR2B. Finally, it was previously reported that the sensitivity of the NMDAR to ethanol in different brain regions depends on intracellular protein compartmentalization (20).

Implications

In summary, these studies suggest the NR2A-containing receptors, but not NR2B, are internalized upon exposure to ethanol. Thus, ethanol treatment leads to a switch of subunit composition of active channels from NR2B- and NR2A-containing NMDARs to mainly NR2B-containing receptors. Subunit compositional switch of functional NMDARs has been previously detected during development and spontaneous synaptic activity (66). Here, it is proposed that exposure of neurons to drugs of abuse such as ethanol also alters the ratio of functional NR2-containing NMDA receptors.

The NMDAR subunits play a role in synaptic plasticity, and a single exposure to drugs of abuse produces NMDAR-dependent synaptic plasticity (67). Therefore, NR2B-mediated NMDAR functions are likely to contribute to the long-lasting synaptic plasticity processes that ultimately result in phenotypes that underlie alcohol abuse., Recently, Liu and colleagues (37) found that NR2A-containing NMDARs mediate long-term potentiation (LTP), whereas the activation of NR2B-containing NMDAR triggers long-term depression (LTD) in the CA1 region of the hippocampus. Interestingly, ethanol exposure was found to inhibit LTP (68), and to enhance LTD (69), raising the possibility that the observed inhibition of LTP is mediated via a decrease in active NR2A-containing channels, and the increase in LTD is mediated via the remaining NR2B-containing channels.

NMDARs are known to play an important role in various phenotypes that contribute to the development of alcohol addiction (5), and studies have suggested that NR2B subunit-selective antagonists may be effective against alcohol dependence (70). It is therefore proposed that the changes in subunit composition of active NMDARs in the therefore of ethanol contribute to the changes in synaptic plasticity that lead to the phenotypes associated with alcohol addiction.

### REFERENCES

1. Malenka, R. C., and Nicoll, R. A. (1993) Trends Neurosci 16, 521-527
2. Cull-Candy, S., Brickley, S., and Farrant, M. (2001) Curr Opin Neurobiol 11, 327-335
3. Coyle, J. T., Tsai, G., and Goff, D. (2003) Ann N Y Acad Sci 1003, 318-327
4. Siggins, G. R., Martin, G., Roberto, M., Nie, Z., Madamba, S., and De Lecea, L. (2003) Ann N Y Acad Sci 1003, 196-211
5. Krystal, J. H., Petrakis, I. L., Mason, G., Trevisan, L., and D'Souza, D. C. (2003) Pharmacol Ther 99, 79-94
6. Krystal, J. H., Petrakis, I. L., Krupitsky, E., Schutz, C., Trevisan, L., and D'Souza, D.C. (2003) Ann N Y Acad Sci 1003, 176-184
7. Prybylowski, K., and Wenthold, R. J. (2004) J Biol Chem 279, 9673-9676
8. Nong, Y., Huang, Y. Q., and Salter, M. W. (2004) Curr Opin Neurobiol 14, 353-361
9. Tovar, K. R., and Westbrook, G. L. (2002) Neuron 34, 255-264
10. Grosshans, D. R., Clayton, D. A., Coultrap, S. J., and Browning, M.) D. (2002) Nat Neurosci 5, 27-33
11. Mu, Y., Otsuka, T., Horton, A. C., Scott, D. B., and Ehlers, M. D. (2003) Neuron 40, 581-594
12. Nong, Y., Huang, Y. Q., Ju, W., Kalia, L. V., Ahmadian, G., Wang, Y. T., and Salter, M. W. (2003) Nature 422, 302-307
13. Wang, Y. T., and Salter, M. W. (1994) Nature 369, 233-235
14. Scott, D. B., Blanpied, T. A., and Ehlers, M. D. (2003) Neuropharmacology 45, 755-767
15. Dunah, A. W., Sirianni, A. C., Fienberg, A. A., Bastia, E., Schwarzschild, M. A., and Standaert, D. G. (2004) Mol Pharmacol 65, 121-129
16. Vissel, B., Krupp, J. J., Heinemann, S. F., and Westbrook, G. L. (2001) Nat Neurosci 4, 587-596
17. Thornton, C., Yaka, R., Dinh, S., and Ron, D. (2003) J Biol Chem
18. Miyakawa, T., Yagi, T., Kitazawa, H., Yasuda, M., Kawai, N., Tsuboi, K., and Niki, H. (1997) Science 278, 698-701
19. Alvestad, R. M., Grosshans, D. R., Coultrap, S. J., Nakazawa, T., Yamamoto, T., and Browning, M. D. (2003) J Biol Chem 278, 11020-11025
20. Yaka, R., Phamluong, K., and Ron, D. (2003) J Neurosci 23, 3623-3632
21. Grimwood, S., Le Bourdelles, B., Atack, J. R., Barton, C., Cockett, W., Cook, S. M., Gilbert, E., Hutson, P. H., McKernan, R. M., Myers, J., Ragan, C. I., Wingrove, P. B., and Whiting, P. J. (1996) J Neurochem 66, 2239-2247.
22. Grosshans, D. R., Clayton, D. A., Coultrap, S. J., and Browning, M.) D. (2002) Sci STKE 137, PL8
23. Lavezzari, G., McCallum, J., Lee, R., and Roche, K. W. (2003) Neuropharmacology 45, 729-737
24. Brambilla, R., Gnesutta, N., Minichiello, L., White, G., Roylance, A. J., Herron, C. E., Ramsey, M., Wolfer, D. P., Cestari, V., Rossi-Arnaud, C., Grant, S. G., Chapman, P. F., Lipp, H. P., Sturani, E., and Klein, R. (1997) Nature 390, 281-286
25. Manabe, T., Aiba, A., Yamada, A., Ichise, T., Sakagami, H., Kondo, H., and Katsuki, M. (2000) J Neurosci 20, 2504-2511
26. Salter, M. W., and Kalia, L. V. (2004) Nat Rev Neurosci 5, 317-328
27. Leon, J., Guerrero, I., and Pellicer, A. (1987) Mol Cell Biol 7, 1535-1540
28. Wadia, J. S., and Dowdy, S. F. (2003) Curr Protein Pept Sci 4, 97-104
29. Feig, L. A., and Cooper, G. M. (1988) Mol Cell Biol 8, 3235-3243
30. Lovinger, D. M., White, G., and Weight, F. F. (1989) Science 243, 1721-1724
31. Lovinger, D. M., White, G., and Weight, F. F. (1990) J Neurosci 10, 1372-1379
32. Morrisett, R. A., Martin, D., Oetting, T. A., Lewis, D. V., Wilson, W. A., and Swartzwelder, H. S. (1991) Neuropharmacology 30, 1173-1178
33. Grover, C. A., Frye, G. D., and Griffith, W. H. (1994) Brain Res 642, 70-76
34. Li, H. F., and Kendig, J. J. (2003) Br J Pharmacol 139, 73-80
35. Ludvig, N., George, M. A., Tang, H. M., Gonzales, R. A., and Bungay, P. M. (2001) Brain Res 900, 252-260.
36. Farrant, M., Feldmeyer, D., Takahashi, T., and Cull-Candy, S. G. (1994) Nature 368, 335-339
37. Liu, L., Wong, T. P., Pozza, M. F., Lingenhoehl, K., Wang, Y., Sheng, M., Auberson, Y. P., and Wang, Y. T. (2004) Science 304, 1021-1024
38. Okabe, S., Miwa, A., and Okado, H. (1999) J Neurosci 19, 7781-7792
39. Ehlers, M. D., Tingley, W. G., and Huganir, R. L. (1995) Science 269, 1734-1737
40. Mirshahi, T., Anders, D. L., Ronald, K. M., and Woodward, J. J. (1998) J Neurochem 71, 1095-1107
41. Anders, D. L., Blevins, T., Smothers, C. T., and Woodward, J. J. (2000) J Biol Chem 275, 15019-15024
42. Honse, Y., Nixon, K. M., Browning, M. D., and Leslie, S. W. (2003) Neuroscience 122, 689-698
43. Ambrosini, A., Tininini, S., Barassi, A., Racagni, G., Sturani, E., and Zippel, R. (2000) Brain Res Mol Brain Res 75, 54-60
44. Dumaz, N., and Marais, R. (2003) J Biol Chem 278, 29819-29823
45. Diamond, I., and Gordon, A. S. (1997) Physiol Rev 77, 1-20
46. Huang, E. J., and Reichardt, L. F. (2001) Annu Rev Neurosci 24, 677-736
47. McGough, N. N., He, D. Y., Logrip, M. L., Jeanblanc, J., Phamluong, K., Luong, K., Kharazia, V., Janak, P. H., and Ron, D. (2004) J Neurosci 24, 10542-10552
48. Kornau, H. C., Schenker, L. T., Kennedy, M. B., and Seeburg, P. H. (1995) Science 269, 1737-1740
49. Roche, K. W., Standley, S., McCallum, J., Dune Ly, C., Ehlers, M. D., and Wenthold, R. J. (2001) Nat Neurosci 4, 794-802
50. Ron, D. (2004) Neuroscientist 10, 325-336
51. Fang, M., Tao, Y. X., He, F., Zhang, M., Levine, C. F., Mao, P., Tao, F., Chou, C. L., Sadegh-Nasseri, S., and Johns, R. A. (2003) J Biol Chem 278, 36669-36675
52. Kumari, M., and Ticku, M. K. (2000) Prog Drug Res 54, 152-189
53. Allgaier, C. (2002) Neurochem Int 41, 377-382
54. Monyer, H., Burnashev, N., Laurie, D. J., Sakmann, B., and Seeburg, P. H. (1994) Neuron 12, 529-540
55. Blevins, T., Mirshahi, T., Chandler, L. J., and Woodward, J. J. (1997) J Neurochem 69, 2345-2354
56. Anders, D. L., Blevins, T., Sutton, G., Chandler, L. J., and Woodward, J. J. (1999) Alcohol Clin Exp Res 23, 357-362
57. Ronald, K. M., Mirshahi, T., and Woodward, J. J. (2001) J Biol Chem 276, 44729-44735
58. Smothers, C. T., Clayton, R., Blevins, T., and Woodward, J. J. (2001) Neurochem Int 38, 333-340
59. Lovinger, D. M. (1995) J Pharmacol Exp Ther 274, 164-172
60. Popp, R. L., Lickteig, R., Browning, M. D., and Lovinger, D. M. (1998) Neuropharmacology 37, 45-56
61. Kuner, T., Schoepfer, R., and Korpi, E. R. (1993) Neuroreport 5, 279-300
62. Chu, B., Anantharam, V., and Treistman, S. N. (1995) J Neurochem 65, 140-148
63. Masood, K., Wu, C., Brauneis, U., and Weight, F. F. (1994) Mol Pharmacol 45, 324-329
64. Mirshahi, T., and Woodward, J. J. (1995) Neuropharmacology 34, 347-355
65. Popp, R. L., Lickteig, R. L., and Lovinger, D. M. (1999) J Pharmacol Exp Ther 289, 1564-1574
66. Barria, A., and Malinow, R. (2002) Neuron 35, 345-353
67. Ungless, M. A., Whistler, J. L., Malenka, R. C., and Bonci, A. (2001) Nature 411, 583-587
68. Morrisett, R. A., and Swartzwelder, H. S. (1993) J Neurosci 13, 2264-2272
69. Hendricson, A. W., Miao, C. L., Lippmann, M. J., and Morrisett, R. A. (2002) J Pharmacol Exp Ther 301, 938-944
70. Nagy, J. (2004) Curr Drug Targets CNS Neurol Disord 3, 169-179

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose.

1. A method of mitigating a symptom of maladaptive substance use, the method comprising administering an effective amount of an inhibitor of H-ras to a subject, whereby the symptom of maladaptive substance use is mitigated.

2. The method of claim 1, wherein the substance comprises a drug selected from the group consisting of an opioid, a psychostimulant, a cannabinoid, an empathogen, a dissociative drug, and alcohol.

3. The method of claim 1, wherein the inhibitor comprises an antagonist that inhibits a function of H-ras.

4. The method of claim 1, wherein the symptom of maladaptive substance use is selected from the group comprising: drug reward, incentive salience for a drug, drug craving, drug seeking, and drug consumption.

5. The method of claim 1, wherein the inhibitor is administered via implantation of a depot formulation comprising the inhibitor.

6. A pharmaceutical composition comprising an inhibitor of H-ras; and a pharmaceutically acceptable carrier.

7. The pharmaceutical composition of claim 6, wherein the inhibitor comprises an antagonist that inhibits a function of H-ras.

8. The pharmaceutical composition of claim 6, wherein the pharmaceutical composition comprises a depot formulation.

9. A method of prescreening for an agent that can modulate a symptom of maladaptive substance use in a subject, the method comprising:
(a) contacting a test agent with a H-ras;
(b) determining whether the test agent specifically binds to H-ras; and
(c) if the test agent specifically binds to H-ras, selecting the test agent as a potential modulator of a symptom of maladaptive substance use in a subject.

10. A method of prescreening for an agent that can modulate a symptom of maladaptive substance use in a subject, the method comprising:
(a) contacting a test agent with a polynucleotide encoding H-ras;
(b) determining whether the test agent specifically binds to the polynucleotide encoding H-ras; and
(c) if the test agent specifically binds to the polynucleotide encoding H-ras, selecting the test agent as a potential modulator of a symptom of maladaptive substance use in a subject.

11. (canceled)

12. (canceled)

13. A method of screening for an agent that can modulate a symptom of maladaptive substance use in a subject, the method comprising:
(a) contacting a test agent with a H-ras,
(b) determining whether the test agent acts as an agonist or an antagonist of H-ras;
(c) if the test agent acts as an agonist or antagonist of H-ras, selecting the test agent as a potential modulator of maladaptive substance use in a subject.

14. (canceled)

15. (canceled)

16. (canceled)

17. (canceled)

18. (canceled)

19. (canceled)

20. (canceled)

21. (canceled)

22. (canceled)

23. A method of screening for an agent that that can modulate maladaptive substance use in a subject, the method comprising:
(a) selecting a modulator of a H-ras as a test agent; and
(b) measuring the ability of the selected test agent to modulate a symptom of maladaptive substance use in an animal model.

24. (canceled)

25. (canceled)

26. (canceled)

27. A method of assessing a subject's risk for maladaptive substance use, the method comprising determining the level of H-ras polypeptides, polynucleotides, or function in a biological sample from the subject, wherein risk for maladaptive substance use is directly correlated with said level.

28. (canceled)

29. A kit comprising:
(a) an inhibitor of a H-ras in a pharmaceutically acceptable carrier;
(b) instructions for carrying out the method of claim 1.

30. A diagnostic kit comprising:
(a) a component that specifically binds to a H-ras polypeptide or polynucleotide; and
(b) instructions for carrying out the method of claim 27.
